(19) United States
(12) Patent Application Publication
(10) Pub. No.: US 2022/0002793 A1
(43) Pub. Date: Jan. 6, 2022

(54) TAGMENTATION-ASSOCIATED MULTIPLEX PCR ENRICHMENT SEQUENCING

(71) Applicants: AKERSHUS UNIVERSITETSSYKEHUS HF, Lørenskog (NO); KREFTREGISTERET, Majorstuen (NO); UNIVERSITY OF HELSINKI, Helsinki (FI); OSLOMET - STORBYUNIVERSITETET, Oslo (NO)

(72) Inventors: Trine ROUNGE, Eiksmarka (NO); Irene KRAUS CHRISTIANSEN, Hosle (NO); Ole Herman AMBUR, Oslo (NO); Sonja LAGSTRÖM, Lørenskog (NO); Roger MEISAL, Ålesund (NO); Pekka ELLONEN, Tuusula (FI); Maja LEPISTÖ, Helsinki (FI)

(21) Appl. No.: 17/292,958

(22) PCT Filed: Nov. 20, 2019
(86) PCT No.: PCT/IB2019/001254

§ 371 Date: May 11, 2021

(30) Foreign Application Priority Data
Nov. 21, 2018 (NL) 2022043

(51) Int. Cl. C12Q 1/6855; C12Q 1/6806

(52) U.S. Cl. CPC C12Q 1/6855; C12Q 2521/50; C12Q 2521/107; C12Q 1/6806

(57) ABSTRACT

The present invention is related to methods for parallel sequencings of nucleic acid target sequences of interest, and in particular to massively parallel sequencing of nucleic acid sequences such as viral sequences that may have been integrated into a genome. For example, the methods, systems and kits provided herein may be used to enrich and sequence viral DNA sequences such as HPV and HIV sequences.

### FIELD OF THE INVENTION

The present invention is related to methods for parallel sequencing of nucleic acid target sequences of interest, and in particular to massively parallel sequencing of nucleic acid sequences such as viral sequences that have been integrated into a host genome.

### BACKGROUND OF THE INVENTION

Human papillomavirus (HPV) is the main cause of cervical cancer¹, one of the most common cancers in women worldwide, causing more than 200,000 deaths each year^{2,3}. A persistent infection with HPV high-risk genotypes is recognized as a necessary cause of cancer development⁴. Of the 13 carcinogenic high-risk types, HIPV16 and 18 are associated with about 70% of all cervical cancers^{5,6}. HPV infection is also associated with cancer in penis, vulva, vagina, anus, and head and neck⁷. However, only a small fraction of HPV infections at any site will progress to cancer⁸. This indicates that in addition to HPV infection, additional factors such as HPV genomic variability and chromosomal integration, could contribute to the HPV-induced carcinogenic process. An appropriate sequencing approach is needed to uncover these genomic events during a persistent HPV infection.

HPV contains an approximately 7.9 kb circular double-stranded DNA genome, consisting of early region genes (E1, E2, E4-7), late region genes (L1, L2) and an upstream regulatory region (URR)⁹. To date, more than 200 HPV types have been identified¹⁰. Each individual HPV type shares at least 90% sequence identity in the conserved L1 open reading frame (ORF) nucleotide sequence. Isolates of the same HPV types that differ by 1-10% or 0.5-1% across the genome are referred to as variant lineages or sublineages, respectively^{11,12} Despite phylogenetic relatedness, HPV variant lineages can differ in their carcinogenic potential¹³⁻¹⁶. Traditionally, studies have focused on cancer risk of main variants. However, recent studies have revealed variability below the level of variant lineages that may be evidence of intra-host viral evolution and adaptation⁷⁻²⁰. In contrast to a limited number of studies on HPV variability, HPV integration into the host genome has been more widely studied and is regarded as a determining event in cervical carcinogenesis²¹⁻²³. Upon integration, disruption or complete deletion of the E1 or E2 gene is often observed in cancers, having caused constitutive expression of the E6 and E7 oncogenes²⁴⁻²⁶, inactivation of cell cycle checkpoints and genetic instability²³. Viral integration may also lead to modified expression of cellular genes nearby, disruption of genes, as well as genomic amplifications that may promote oncogenesis^{23,27}. The finding of certain chromosomal clusters of integration in precancerous lesions and cancers²⁸ also suggests a selective advantage of specific HPV integrations. Still, several important questions remain for HPV integration and more comprehensive analyses of integration sites are needed in order to expand our understanding of HPV pathogenesis.

The development of next generation sequencing (NGS) technologies has provided new tools for viral genomic research. During the recent years, a few studies have described different NGS based approaches to study HPV variability and integration in the human genome. The most common approaches used in HPV genomic analyses are based on target enrichment using highly multiplexed degenerate primers²⁹, enrichment by multiplex PCR using HPV16 forward primers³⁰, bead-based target capture³¹⁻³³ and rolling circle amplification³⁴ followed by NGS. These methods are however designed to detect either HPV integration or HPV variability. In addition, target capture methods poorly enrich HPV and remain expensive due to high probe cost and off-target sequencing.

### SUMMARY OF THE INVENTION

The present invention is related to methods for parallel sequencing of nucleic acid target sequences of interest, and in particular to massively parallel sequencing of nucleic acid sequences such as viral sequences that have been inserted into the host genome.

In some embodiments, the present invention provides methods of amplifying a target nucleic acid sequence for use in a parallel sequencing method comprising: tagmenting a target nucleic sample to provide a plurality of tagmented sequences comprising a transposon adapter sequence at the ends of the tagmented sequences; contacting a first sample of the tagmented sequences with 1) a tag primer comprising a tag sequence portion that anneals to the transposon adapter sequence and a tag primer adapter portion, 2) a plurality of forward primers, each forward primer comprising a target sequence portion that anneals to a preselected portion of the sense strand of the target nucleic acid sequence and a forward primer sequencing portion, and 3) a sequencing primer comprising a portion that anneals to the forward sequencing portion of the forward primer and a sequencing primer adapter portion; performing a forward amplification reaction on the first sample of the tagmented sequences to provide a first library of amplicons spanning the target nucleic acid sequence; contacting a second sample of the tagmented sequences with 1) a tag primer comprising a tag sequence portion that anneals to the transposon sequence and a tag primer adapter portion, 2) a plurality of reverse primers, each reverse primer comprising a target sequence portion that anneals to a preselected portion of the antisense strand of the target nucleic acid sequence and a reverse primer sequencing portion, and 3) a sequencing primer comprising a portion that anneals to the reverse sequencing portion of the forward primer and a sequencing primer adapter portion; and performing a reverse amplification reaction on the second sample of the tagmented sequences to provide a second library of amplicons spanning the target nucleic acid sequence.

In some preferred embodiments, the tag primer adapter portion of the tag primer comprises a barcode sequence and a first tail portion. In some preferred embodiments, the barcode sequence is an Illumina i5 index sequence and the first tail portion is an Illumina p5 tail. In some preferred embodiments, the barcode sequence is an Illumina i7 index sequence and the first tail portion is an Illumina p7 tail. In some preferred embodiments, the sequencing primer adapter portion of the sequencing primer comprises a barcode sequence and a second tail portion. In some preferred embodiments, the barcode sequence is an Illumina i7 index sequence and the second tail portion is an Illumina p7 tail. In some preferred embodiments, the barcode sequence is an Illumina i5 index sequence and the second tail portion is an Illumina p5 tail. In some preferred embodiments, the tag primers used in the forward and reverse reactions are identical. In some preferred embodiments, the sequencing primers used in the forward and reverse reactions are identical. In some preferred embodiments, the tag primers comprise an Illumina i5 index sequence and an Illumina p5 tail and the sequencing primers comprise an Illumina i7 index sequence and an Illumina p7 tail. In some preferred embodiments, the tag primers comprise an Illumina i7 index sequence and an Illumina p7 tail and the sequencing primers comprise an Illumina i5 index sequence and an Illumina p5 tail.

In some preferred embodiments, the plurality of forward primers comprises from about 10 to 500 forward primers. In some preferred embodiments, the plurality of forward primers are designed so that the primers anneal to the target nucleic acid sequence at intervals of from 50 to 500 bases along the entire length of the target nucleic acid sequence. In some preferred embodiments, the plurality of reverse primers comprises from about 10 to 500 forward primers. In some preferred embodiments, the plurality of reverse primers are designed so that the primers anneal to the target nucleic acid sequence at intervals of from 50 to 500 bases along the entire length of the target nucleic acid sequence.

In some preferred embodiments, the target nucleic sequence is from 1000 to 100000 bases in length. In some preferred embodiments, the target nucleic sequence is an integrated viral sequence. In some preferred embodiments, the integrated viral sequence is Human Papillomavirus (HPV) sequence. In some preferred embodiments, the integrated viral sequence is a Human Immunodeficiency Virus (HIV).

In some preferred embodiments, the tagmentation reaction produces fragments that span the 5′- and 3′-integration sites of the integrated viral sequence so that after amplification the library contains amplicons that span the 5′- and 3′-integration sites of the integrated viral sequence.

In some preferred embodiments, the methods further comprise the step of sequencing the libraries of amplicons. In some preferred embodiments, the libraries are pooled for sequencing. In some preferred embodiments, the libraries are sequenced by massively parallel sequencing.

In some embodiments, the present invention provides kits or systems for amplifying tagmented target nucleic acid tagged with transposon adapter sequence in preparation for sequencing comprising: a tag primer comprising a tag sequence portion that anneals to the transposon adapter sequence and a tag primer adapter portion, a plurality of forward primers, each forward primer comprising a target sequence portion that anneals to a preselected portion of the sense strand of the target nucleic acid sequence and a forward primer sequencing portion, a plurality of reverse primers, each reverse primer comprising a target sequence portion that anneals to a preselected portion of the antisense strand of the target nucleic acid sequence and a reverse primer sequencing portion, a sequencing primer comprising a portion that anneals to the forward and reverse sequencing portion of the forward and reverse primer and a sequencing primer adapter portion.

In some preferred embodiments, the tag primer adapter portion of the tag primer comprises a barcode sequence and a first tail portion. In some preferred embodiments, the barcode sequence is an Illumina i5 index sequence and the first tail portion is an Illumina p5 tail. In some preferred embodiments, the barcode sequence is an Illumina i7 index sequence and the first tail portion is an Illumina p7 tail. In some preferred embodiments, the sequencing primer adapter portion of the sequencing primer comprises a barcode sequence and a second tail portion. In some preferred embodiments, the barcode sequence is an Illumina i7 index sequence and the second tail portion is an Illumina p7 tail. In some preferred embodiments, the barcode sequence is an Illumina i5 index sequence and the second tail portion is an Illumina p5 tail. In some preferred embodiments, the tag primers used in the forward and reverse reactions are identical. In some preferred embodiments, the sequencing primers used in the forward and reverse reactions are identical. In some preferred embodiments, the tag primers comprise an Illumina i5 index sequence and an Illumina p5 tail and the sequencing primers comprise an Illumina i7 index sequence and an Illumina p7 tail. In some preferred embodiments, the tag primers comprise an Illumina i7 index sequence and an Illumina p7 tail and the sequencing primers comprise an Illumina i5 index sequence and an Illumina p5 tail.

In some preferred embodiments, the plurality of forward primers comprises from about 10 to 500 forward primers. In some preferred embodiments, the plurality of forward primers are designed so that the primers anneal to the target nucleic acid sequence at intervals of from 50 to 500 bases along the entire length of the target nucleic acid sequence. In some preferred embodiments, the plurality of reverse primers comprises from about 10 to 500 forward primers. In some preferred embodiments, the plurality of reverse primers are designed so that the primers anneal to the target nucleic acid sequence at intervals of from 50 to 500 bases along the entire length of the target nucleic acid sequence.

In some preferred embodiments, the target nucleic sequence is from 1000 to 100000 bases in length. In some preferred embodiments, the target nucleic sequence is an integrated viral sequence. In some preferred embodiments, the integrated viral sequence is Human Papillomavirus (HPV) sequence. In some preferred embodiments, the integrated viral sequence is a Human Immunodeficiency Virus (HIV).

In some preferred embodiments, the kit or system further comprises a transposase. In some preferred embodiments, the kit or system further comprises a polymerase. In some preferred embodiments, the kit or system further comprises one or more buffers for reactions using the transposase or polymerase.

### DESCRIPTION OF THE DRAWINGS

FIG. 1. Primer design, laboratory and bioinformatics workflows of the TaME-seq method.

FIG. 2. HPV genome sequencing coverage in HPV positive samples. The coverage plots of a) CaSki, b) HeLa, c) LBC34, d) LBC11, and e) MS751 are aligned to the respective target HPV genomes. The location of early (E1, E2, E4-7), late (L1, L2) genes, URR, and forward (red arrows) and reverse (blue arrows) HPV primers is indicated below the genomic positions.

FIG. 3. An IGV visualisation of HISAT and LAST alignments to find HPV-human integration breakpoints. All the reads were first mapped with HISAT2 and then the unmapped reads were remapped with LAST. a) SiHa reads mapping to chromosome 13 (GRCh38/hg38). Light blue HISAT reads have pairs mapping to HPV16 reference genome. Multi-coloured part of the LAST reads are mismatched bases that map to HPV16 (not visualised). b) SiHa reads mapping to HPV16 reference genome. Orange HISAT reads have pairs mapping to chromosome 13 (GRCh38/hg38). Multi-coloured part of the LAST reads are mismatched bases that map to chr13 (not visualised). Red arrows point to the exact breakpoint positions.

FIG. 4. Number of variable sites in SiHa replicates. SiHa-1 (red dots) and SiHa-2 (blue dots) served as technical replicates to assess the variant calling performance. In SiHa libraries, sequenced on MiSeq and HiSeq 2500 platforms, increasing number of variable sites were detected with higher mean coverage.

FIG. 5. Proportion of variable sites in HPV genes in HPV positive samples. The number of variable sites were normalised by the length of each HPV gene. Gradient green (0% variable sites) to red (30% variable sites) color-coding of the results is shown to present the considerable variability in the samples throughout the HPV genome.

FIG. 6. HPV nucleotide variation observed in two samples. The plots showing variable sites and variant allele frequency (%) in a) CaSki, and b) LBC54 are aligned to the respective target HPV genomes. The location of genes and URR is indicated below the genomic positions. The red line indicates the variant calling threshold value of 0.2%.

FIG. 7. Number of integration breakpoints in HPV16 and HPV18 positive samples with integration. Vertical lines indicate the mean number of integration breakpoints and each dot indicates a sample.

FIG. 8. Integration breakpoints in HPV genes (a) and human chromosomes (b). (a) Expected and observed number of integration breakpoints in HPV genes in HPV16 and HPV18 positive samples. (b) Expected and observed number of integration breakpoints in human chromosomes in HPV16 and HPV18 positive samples.

FIG. 9. Genomic deletions in HPV16 (a) and HPV18 positive samples (b-f). The respective regions were deleted totally (no sequencing coverage) or partially (low sequencing coverage). Dashed vertical lines indicate the integration breakpoints detected in the samples with integration analysis.

FIG. 10. Number of variants and variant frequencies in HPV16 and HPV18 positive samples. (a) Number of variants presented as boxplots across the different diagnosis groups. (b) Variant frequencies (%) of detected minor variants shown across the different diagnosis groups. The vertical bar indicates the mean variant frequency.

FIG. 11. Number of variants, and nonsynonymous and synonymous variations in HPV genomic regions. (a) Heat map with green-yellow-red gradient color-coding representing mean number of variants per sample in HPV16 and HPV18 genomic regions across the different diagnosis groups. (b) Heat map with blue-white-red gradient color-coding representing the ratio of non-synonymous to synonymous substitutions (dN/dS) in HPV16 and HPV18 genomic regions across the different diagnosis groups.

FIG. 12. C>T mutational signatures in HPV16 and HPV18 positive samples. The mean proportion of 16 trinucleotide substitution types is shown across the different diagnosis groups. Error bars represent the standard error of the mean.

### DEFINITIONS

Unless specifically defined or described differently elsewhere herein, the following terms and descriptions related to the invention shall be understood as given below.

When the terms “for example”, “e.g.”, “such as”, “include”, “including” or variations thereof are used herein, these terms will not be deemed to be terms of limitation, and will be interpreted to mean “but not limited to” or “without limitation.” The use of terms “a” and “an” and “the” and similar referents in the context of describing the invention (especially in the context of the following claims) are to be construed to cover both the singular and the plural, unless otherwise indicated herein or clearly contradicted by context.

As used herein the term “nucleic acid” and/or “oligonucleotide” and/or grammatical equivalents thereof can refer to at least two nucleotide monomers linked together. A nucleic acid can generally contain phosphodiester bonds, however, in some embodiments, nucleic acid analogs may have other types of backbones, comprising, for example, phosphoramide (Beaucage, et al., Tetrahedron, 49:1925 (1993); Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986), incorporated by reference in their entireties), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989), incorporated by reference in its entirety), O-methylphosphoroamidite linkages (see Eckstein, Oligonucleotides and Analogues: A Practical Approach, Oxford University Press, incorporated by reference in its entirety), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), incorporated by reference in their entireties).

Other analog nucleic acids include those with positive backbones (Denpcy, et al., Proc. Natl. Acad. Sci. USA, 92:6097 (1995), incorporated by reference in its entirety); non-ionic backbones (U.S. Pat. Nos. 5,386,023; 5,637,684; 5,602,240; 5,216,141; and U.S. Pat. No. 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleosides & Nucleotides, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996), incorporated by reference in their entireties) and non-ribose (U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Coo, incorporated by reference in their entireties). Nucleic acids may also contain one or more carbocyclic sugars (see Jenkins, et al., Chem. Soc. Rev., (1995) pp. 169 176).

Modifications of the ribose-phosphate backbone may be done to facilitate the addition of additional moieties such as labels, or to increase the stability of such molecules under certain conditions. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, for example, genomic or cDNA, RNA or a hybrid. A nucleic acid can contain any combination of deoxyribo- and ribo-nucleotides, and any combination of bases, including uracil, adenine, thymine, cytosine, guanine, inosine, xanthanine, hypoxanthanine, isocytosine, isoguanine, and base analogs such as nitropyrrole (including 3-nitropyrrole) and nitroindole (including 5-nitroindole), etc.

In some embodiments, a nucleic acid can include at least one promiscuous base. Promiscuous bases can base-pair with more than one different type of base. In some embodiments, a promiscuous base can base-pair with at least two different types of bases and no more than three different types of bases. An example of a promiscuous base includes inosine that may pair with adenine, thymine, or cytosine. Other examples include hypoxanthine, 5-nitroindole, acylic 5-nitroindole, 4-nitropyrazole, 4-nitroimidazole and 3-nitropyrrole (Loakes et al., Nucleic Acid Res. 22:4039 (1994); Van Aerschot et al., Nucleic Acid Res. 23:4363 (1995); Nichols et al., Nature 369:492 (1994); Berstrom et al., Nucleic Acid Res. 25:1935 (1997); Loakes et al., Nucleic Acid Res. 23:2361 (1995); Loakes et al., J. Mol. Biol. 270:426 (1997); and Fotin et al., Nucleic Acid Res. 26:1515 (1998), incorporated by reference in their entireties). Promiscuous bases that can base-pair with at least three, four or more types of bases can also be used.

As used herein, the term “nucleotide analog” and/or grammatical equivalents thereof can refer to synthetic analogs having modified nucleotide base portions, modified pentose portions, and/or modified phosphate portions, and, in the case of polynucleotides, modified internucleotide linkages, as generally described elsewhere (e.g., Scheit, Nucleotide Analogs, John Wiley, New York, 1980; Englisch, Angew. Chem. Int. Ed. Engl. 30:613-29, 1991; Agarwal, Protocols for Polynucleotides and Analogs, Humana Press, 1994; and S. Verma and F. Eckstein, Ann. Rev. Biochem. 67:99-134, 1998). Generally, modified phosphate portions comprise analogs of phosphate wherein the phosphorous atom is in the +5 oxidation state and one or more of the oxygen atoms is replaced with a non-oxygen moiety, e.g., sulfur. Exemplary phosphate analogs include but are not limited to phosphorothioate, phosphorodithioate, phosphoroselenoate, phosphorodiselenoate, phosphoroanilothioate, phosphoranilidate, phosphoramidate, boronophosphates, including associated counterions, e.g., H⁺, NH⁴⁺, Na⁺, if such counterions are present. Example modified nucleotide base portions include but are not limited to 5-methylcytosine (5mC); C-5-propynyl analogs, including but not limited to, C-5 propynyl-C and C-5 propynyl-U; 2,6-diaminopurine, also known as 2-amino adenine or 2-amino-dA); hypoxanthine, pseudouridine, 2-thiopyrimidine, isocytosine (isoC), 5-methyl isoC, and isoguanine (isoG; see, e.g., U.S. Pat. No. 5,432,272). Exemplary modified pentose portions include but are not limited to, locked nucleic acid (LNA) analogs including without limitation Bz-A-LNA, 5-Me-Bz-C-LNA, dmf-G-LNA, and T-LNA (see, e.g., The Glen Report, 16(2):5, 2003; Koshkin et al., Tetrahedron 54:3607-30, 1998), and 2′- or 3′-modifications where the 2′- or 3′-position is hydrogen, hydroxy, alkoxy (e.g., methoxy, ethoxy, allyloxy, isopropoxy, butoxy, isobutoxy and phenoxy), azido, amino, alkylamino, fluoro, chloro, or bromo. Modified internucleotide linkages include phosphate analogs, analogs having achiral and uncharged intersubunit linkages (e.g., Sterchak, E. P. et al., Organic Chem., 52:4202, 1987), and uncharged morpholino-based polymers having achiral intersubunit linkages (see, e.g., U.S. Pat. No. 5,034,506). Some internucleotide linkage analogs include morpholidate, acetal, and polyamide-linked heterocycles. In one class of nucleotide analogs, known as peptide nucleic acids, including pseudocomplementary peptide nucleic acids (“PNA”), a conventional sugar and internucleotide linkage has been replaced with a 2-aminoethylglycine amide backbone polymer (see, e.g., Nielsen et al., Science, 254:1497-1500, 1991; Egholm et al., J. Am. Chem. Soc., 114: 1895-1897 1992; Demidov et al., Proc. Natl. Acad. Sci. 99:5953-58, 2002; Peptide Nucleic Acids: Protocols and Applications, Nielsen, ed., Horizon Bioscience, 2004).

As used herein, the term “sequencing read” and/or grammatical equivalents thereof can refer to a repetitive process of physical or chemical steps that is carried out to obtain signals indicative of the order of monomers in a polymer. The signals can be indicative of an order of monomers at single monomer resolution or lower resolution. In particular embodiments, the steps can be initiated on a nucleic acid target and carried out to obtain signals indicative of the order of bases in the nucleic acid target. The process can be carried out to its typical completion, which is usually defined by the point at which signals from the process can no longer distinguish bases of the target with a reasonable level of certainty. If desired, completion can occur earlier, for example, once a desired amount of sequence information has been obtained. A sequencing read can be carried out on a single target nucleic acid molecule or simultaneously on a population of target nucleic acid molecules having the same sequence, or simultaneously on a population of target nucleic acids having different sequences. In some embodiments, a sequencing read is terminated when signals are no longer obtained from one or more target nucleic acid molecules from which signal acquisition was initiated. For example, a sequencing read can be initiated for one or more target nucleic acid molecules that are present on a solid phase substrate and terminated upon removal of the one or more target nucleic acid molecules from the substrate. Sequencing can be terminated by otherwise ceasing detection of the target nucleic acids that were present on the substrate when the sequencing run was initiated.

As used herein, the term “sequencing representation” and/or grammatical equivalents thereof can refer to information that signifies the order and type of monomeric units in the polymer. For example, the information can indicate the order and type of nucleotides in a nucleic acid. The information can be in any of a variety of formats including, for example, a depiction, image, electronic medium, series of symbols, series of numbers, series of letters, series of colors, etc. The information can be at single monomer resolution or at lower resolution, as set forth in further detail below. An exemplary polymer is a nucleic acid, such as DNA or RNA, having nucleotide units. A series of “A,” “T,” “G,” and “C” letters is a well-known sequence representation for DNA that can be correlated, at single nucleotide resolution, with the actual sequence of a DNA molecule. Other exemplary polymers are proteins having amino acid units and polysaccharides having saccharide units.

As used herein the term “at least a portion” and/or grammatical equivalents thereof can refer to any fraction of a whole amount. For example, “at least a portion” can refer to at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, 99.9% or 100% of a whole amount.

The invention also comprises kits and individual compositions for any of the methods of the invention. A kit is a combination of individual compositions useful for carrying out a method of the invention, wherein the compositions are optimized for use together in the method. A composition comprises an individual component or a blend of components for at least one step of a method of the invention. The invention comprises any kit that can be assembled from a combination of any two compositions of the invention, and any novel composition that is used in a kit or method of the invention. Alternatively, a kit may be assembled from a single component or composition in a convenient use format, e.g., pre-aliquoted in single use portion, and may optionally include a set of instructions for use of the component or composition.

### DETAILED DESCRIPTION OF THE INVENTION

The present invention is related to methods for parallel sequencing of nucleic acid target sequences of interest, and in particular, to massively parallel sequencing of nucleic acid sequences such as viral sequences, including sequences integrated into another genome, episomal sequences, and other nucleic acid and genomic sequences. In some preferred embodiments, the methods, systems and kits provided herein are particularly useful for enriching and sequencing target nucleic sequences such as viral sequences (e.g., HPV or HIV sequences) that may have been integrated into a genome.

The methods, systems and kits and the present invention preferably utilize components of established massively parallel sequencing technologies such as those provided by Illumina. Suitable sequencing technologies for use in the present invention include, but are not limited, to those described in US Pat. Publ. 20100120098, US Pat. Publ. 20120208705, US Pat. Publ. 20120208724, WO2012/061832, and US Pat. Publ. 2015/0368638, each of which is incorporated herein by reference in its entirety.

In some preferred embodiments, the systems, methods and kits for sequencing a target nucleic acid sequence comprise methods and reagents for tagmenting a sample of nucleic acid (e.g., genomic DNA). Suitable tagmentation reagents include, for example, those provided by Illumina in the NEXTERA DNA or NEXTERA DNA Flex library preparation kit. The transposomes are utilized to fragment the nucleic acid samples at approximately 250 to 1,500 bp in length, more preferably from 200 to 400 bp intervals and most preferably at about 300 bp intervals. As part of the tagmentation reaction, transposon adapter sequences are added to the 5′ ends of the sequence fragments. In the normal Nextera protocol, indexed sequencing primers that anneal to the adapter sequences are used in a limited cycle PCR to amplify the fragments to make a library for sequencing.

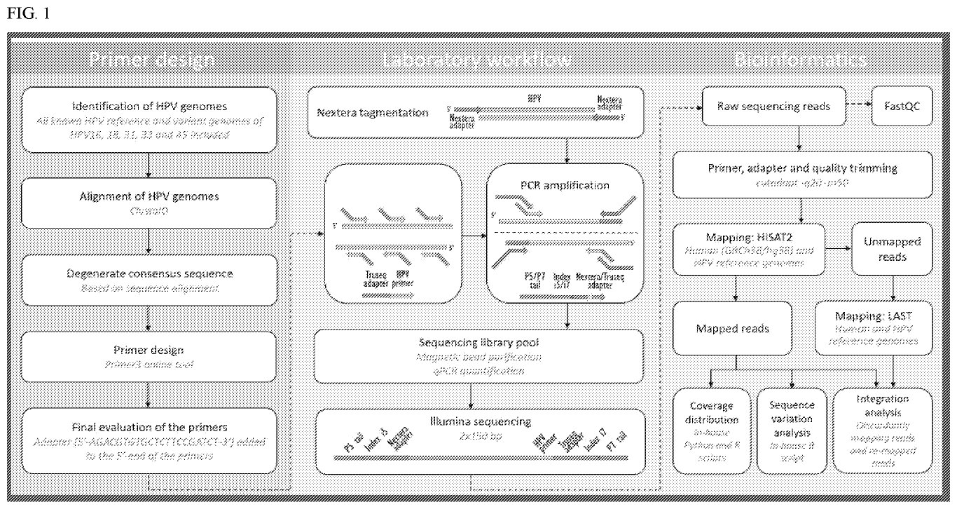

In contrast, the protocols of the present invention preferably call for dividing the tagmented nucleic acid sample into two pools, a forward pool and a reverse pool. See FIG. 1. In some embodiments, the forward and reverse pools are utilized in a multiplex PCR conducted under conditions so that the target DNA sequence, for example, an integrated HPV or HIV sequence, is enriched for subsequent sequencing. As shown in FIG. 1, the multiplex reactions utilize a tag primer that anneals to the transposon adapter sequence on one end of the DNA fragment pool, a set of forward or reverse primers that are specifically designed to anneal to the target DNA sequence and which includes a tail portion (denoted the Truseq adapter in the FIG. 1) compatible with the sequencing primer, and a sequencing primer that anneals to a tail portion of the forward or reverse primers. Preferably, the tag and sequencing primers are Illumina Truseq™ primers or other similar compatible primers. It will be understood that various combinations of tails such as P5 and P7 tails and index (i.e., barcode) sequences may be utilized. For example, where a P5 tail and i5 index are utilized in the tag primer that binds to the transposon adapter, a P7 tail and i7 index are utilized in the sequencing primer that anneals to the forward or reverse primer. As another example, where a P7 tail and i7 index are utilized in the tag primer that binds to the transposon adapter, a P5 tail and i5 index are utilized in the sequencing primer that anneals to the forward or reverse primer. In some embodiments, the tag primer is the same for both the forward and the reverse reactions. While preferred embodiments of the present invention utilize primers and reagents that are compatible with Illumina systems, it will be understood by those of skill in the art that other index and tail sequences may be utilized.

The forward and reverse primer sets are preferably designed so that the primers anneal at intervals on the target sequence of from 50 to 500 bases, preferably from 100 to 400 bases, more preferably from 200 to 400 bases and most preferably about 300 bases. The target sequence may be from about 1000 to 100000 bases in length, preferably from about 3000 to 50000 bases in length, and most preferably from about 3000 to 12000 bases in length. In some preferred embodiments, typical forward and reverse primer pools will comprise from about 5 forward or reverse primers to 100 for or reverse primers as are needed to span the target sequence. The multiplex PCR reaction preferably results in amplification of a library of sequences that is enriched for sequences spanning the target DNA sequence as compared to other regions of the genome. The enrichment of these sequences greatly reduces the amount of sequencing needed to study or provide information about the target sequence and allows many target sequences from different sources or that are located in different areas of the genome (e.g., at multiple insertion sites) to be analyzed in parallel. In some preferred embodiments, the protocols of the present invention allow identification of genomic integration sites, for example viral integration sites. It will be understood that when the genomic samples are tagmented, that some of the fragments will span the 5′ or 3′ integration sites of a virus. Thus, when the target DNA is integrated viral DNA and forward and reverse primer sets are utilized that are specific for the viral DNA, the library of amplified fragments will include fragments that include both inserted viral DNA and genomic DNA. Sequencing of the library of fragments therefore allows identification of integration sites.

Following amplification, the libraries may then be sequenced as is known in the art, for example by utilizing the Illumina sequencing reagents. However, as will be apparent to those of skill in the art, other sequencing systems may be utilized.

Some general features of the invention will now be described.

Some embodiments provided herein include transposon sequences. In some embodiments, a transposon sequence includes at least one transposase recognition site and at least one barcode. In some embodiments, a transposon sequence includes a first transposon recognition site, a second transposon recognition site, and a barcode disposed therebetween.

A transposase recognition site can include two complementary nucleic acid sequences, e.g., a double-stranded nucleic acid or a hairpin nucleic acid, that comprise a substrate for a transposase or integrase. The transposase or integrase may bind to the transposase recognition site and insert the transposase recognition site into a target nucleic acid. In some such insertion events, one strand of the transposase recognition site may be transferred into the target nucleic acid.

In some embodiments a transposase recognition site is a component of a transposition system. A transposition system can include a transposase enzyme and a transposase recognition site. In some such systems, the transposase can form a functional complex with a transposes recognition site that is capable of catalyzing a transposition reaction. Some embodiments can include the use of a hyperactive Tn5 transposase and a Tn5-type transposase recognition site (Goryshin, I. and Reznikoff, W. S., J. Biol. Chem., 273: 7367, 1998), or MuA transposase and a Mu transposase recognition site comprising R1 and R2 end sequences (Mizuuchi, K., Cell, 35: 785, 1983; Savilahti, H, et al., EMBO J., 14: 4893, 1995). An exemplary transposase recognition site that forms a complex with a hyperactive Tn5 transposase (e.g., EZ-Tn5™ Transposase, EPICENTRE Biotechnologies, Madison, Wis., USA) comprises the following transferred strand and non-transferred strands: 5′ AGATGTGTATAAGAGACAG 3′, (SEQ ID NO: 1), 5′ CTGTCT CTTATACACATCT 3′ (SEQ ID NO: 2), respectively. More examples of transposition systems that can be used with certain embodiments provided herein include Staphylococcus aureus Tn552 (Colegio O R et al., J. Bacteriol., 183: 2384-8, 2001; Kirby C et al., Mol. Microbiol., 43: 173-86, 2002), Ty1 (Devine S E, and Boeke J D., Nucleic Acids Res., 22: 3765-72, 1994 and International Patent Application No. WO 95/23875), Transposon Tn7 (Craig, N L, Science. 271: 1512, 1996; Craig, N L, Review in: Curr Top Microbiol Immunol., 204: 27-48, 1996), Tn/O and IS10 (Kleckner N, et al., Curr Top Microbiol Immunol., 204: 49-82, 1996), Mariner transposase (Lampe D J, et al., EMBO J., 15: 5470-9, 1996), Tc1 (Plasterk R H, Curr Top Microbiol Immunol, 204: 125-43, 1996), P Element (Gloor, G B, Methods Mol. Biol., 260: 97-114, 2004), Tn3 (Ichikawa H, and Ohtsubo E., J. Biol. Chem. 265: 18829-32, 1990), bacterial insertion sequences (Ohtsubo, F and Sekine, Y, Curr. Top. Microbiol. Immunol. 204: 1-26, 1996), retroviruses (Brown P O, et al., Proc Natl Acad Sci USA, 86: 2525-9, 1989), and retrotransposon of yeast (Boeke J D and Corces V G, Annu Rev Microbiol. 43: 403-34, 1989), the disclosures of which are incorporated herein by reference in their entireties.

Generally, a barcode can include one or more nucleotide sequences that can be used to identify one or more particular nucleic acids. A barcode can comprise at least about 1, 2, 3, 4, 5, 6, 7, 8, 9, 10, 11, 12, 13, 14, 15, 16, 17, 18, 19, 20 or more consecutive nucleotides. In some embodiments, a barcode comprises at least about 10, 20, 30, 40, 50, 60, 70 80, 90, 100 or more consecutive nucleotides. In some embodiments, at least a portion of the barcodes in a population of nucleic acids comprising barcodes is different. In some embodiments, at least about 10%, 20%, 30%, 40%, 50%, 60%, 70%, 80%, 90%, 95%, 99% of the barcodes are different. In more such embodiments, all of the barcodes are different. The diversity of different barcodes in a population of nucleic acids comprising barcodes can be randomly generated or non-randomly generated.

In some embodiments, a transposon sequence comprises at least one barcode. In some embodiments, a transposon sequence comprises a barcode comprising a first barcode sequence and a second barcode sequence. In some such embodiments, the first barcode sequence can be identified or designated to be paired with the second barcode sequence. For example, a known first barcode sequence can be known to be paired with a known second barcode sequence using a reference table comprising a plurality of first and second bar code sequences known to be paired to one another. In another example, the first barcode sequence can comprise the same sequence as the second barcode sequence. In another example, the first barcode sequence can comprise the reverse complement of the second barcode sequence.

In some embodiments, a population of nucleic acids can comprise nucleic acids that include a first barcode sequence and second barcode sequence. In some such embodiments the first and second barcode sequences of a particular nucleic acid can be different. As will be described further herein, paired first and second barcode sequences can be used to identify different nucleic acids comprising barcodes linked with one another.

Some embodiments include transposon sequences comprising a first barcode sequence and a second barcode sequence having a linker disposed therebetween. In other embodiments, the linker can be absent, or can be the sugar-phosphate backbone that connects one nucleotide to another. The linker can comprise, for example, one or more of a nucleotide, a nucleic acid, a non-nucleotide chemical moiety, a nucleotide analogue, amino acid, peptide, polypeptide, or protein. In preferred embodiments, a linker comprises a nucleic acid. The linker can comprise at least about 1, 2, 3, 4, 5, 6, 7, 8, 9, 10, 11, 12, 13, 14, 15, 16, 17, 18, 19, 20, or more nucleotides. In some embodiments, a linker can comprise at least about 10, 20, 30, 40, 50, 60, 70, 80, 90, 100, or more nucleotides.

In some embodiments, the linker can comprise a fragmentation site. A fragmentation site can be used to cleave the physical, but not the informational association between a first barcode sequence and a second barcode sequence. Cleavage may be by biochemical, chemical or other means. In some embodiments, a fragmentation site can include a nucleotide or nucleotide sequence that may be fragmented by various means. For example, a fragmentation site may be a substrate for an enzyme, such as a nuclease, that will cleave the physical association between a first barcode sequence and a second barcode sequence. For example, the fragmentation site comprises a restriction endonuclease site and may be cleaved with an appropriate restriction endonuclease. In another example, a fragmentation site can comprise at least one ribonucleotide in a nucleic acid that may otherwise comprise deoxyribonucleotides and may be cleaved with an RNAse. Chemical cleavage agents capable of selectively cleaving the phosphodiester bond between a deoxyribonucleotide and a ribonucleotide include metal ions, for example rare-earth metal ions (e.g., La.sup.3+, particularly Tm.sup.3+, Yb.sup.3+ or Lu.sup.3+ (Chen et al. Biotechniques. 2002, 32: 518-520; Komiyama et al. Chem. Commun. 1999, 1443-1451)), Fe(3) or Cu(3), or exposure to elevated pH, e.g., treatment with a base such as sodium hydroxide. As used herein, selective cleavage of the phosphodiester bond between a deoxyribonucleotide and a ribonucleotide can refer to the chemical cleavage agent is not capable of cleaving the phosphodiester bond between two deoxyribonucleotides under the same conditions.

In another example, the fragmentation site can comprise one or more recognition sequences for a nickase, that is, a nicking endonuclease that breaks one strand of a double-stranded nucleic acid. Thus, the fragmentation site can comprise a first nickase recognition sequence, a second nickase recognition sequence. The cut site for each recognition sequence can be the same site or different site.

In another example, a fragmentation site can include one or more nucleotide analogues that comprise an abasic site and permits cleavage at the fragmentation site in the presence of certain chemical agents, such as polyamine, N,N′-dimethylethylenediamine (DMED) (U.S. Patent Application Publication No. 2010/0022403, incorporated by reference herein). In some embodiments, an abasic site may be created within a fragmentation site by first providing a fragmentation site comprising a deoxyuridine (U) of a double stranded nucleic acid. The enzyme uracil DNA glycosylase (UDG) may then be used to remove the uracil base, generating an abasic site on one strand. The polynucleotide strand including the abasic site may then be cleaved at the abasic site by treatment with endonuclease (e.g. Endo IV endonuclease, AP lyase, FPG glycosylase/AP lyase, Endo VIII glycosylase/AP lyase), heat or alkali. Abasic sites may also be generated at nucleotide analogues other than deoxyuridine and cleaved in an analogous manner by treatment with endonuclease, heat or alkali. For example, 8-oxo-guanine can be converted to an abasic site by exposure to FPG glycosylase. Deoxyinosine can be converted to an abasic site by exposure to AlkA glycosylase. The abasic sites thus generated may then be cleaved, typically by treatment with a suitable endonuclease (e.g. Endo IV, AP lyase). (U.S. Patent Application Publication No. 2011/0014657, incorporated by reference herein in its entirety).

In another example, a fragmentation site may include a diol linkage which permits cleavage by treatment with periodate (e.g., sodium periodate). In another example, a fragmentation site may include a disulphide group which permits cleavage with a chemical reducing agent, e.g. Tris (2-carboxyethyl)-phosphate hydrochloride (TCEP).

In some embodiments, a fragmentation site may include a cleavable moiety that may be subject to photochemical cleavage. Photochemical cleavage encompasses any method which utilizes light energy in order to achieve cleavage of a nucleic acids, for example, one or both strands of a double-stranded nucleic acid molecule. A site for photochemical cleavage can be provided by a non-nucleotide chemical moiety in a nucleic acid, such as phosphoamidite (4-(4,4′-Dimethoxytrityloxy)butyramidomethyl)-1-(2-nitrophenyl)-ethyl]-2-cyanoethyl-(N,N-diisopropyl)-phosphoramidite) (Glen Research, Sterling, Va., USA, Cat No. 10-4913-XX).

In some embodiments, a fragmentation site can include a peptide, for example, conjugate structure in which a peptide molecule is linked to a nucleic acid. The peptide molecule can subsequently be cleaved by a peptidase enzyme of the appropriate specificity, or any other suitable means of non-enzymatic chemical or photochemical cleavage. In some embodiments, a conjugate between peptide and nucleic acid will be formed by covalently linking a peptide to a nucleic acid, e.g., a strand of a double-stranded nucleic acid. Conjugates between a peptide and nucleic acid can be prepared using techniques generally known in the art. In one such technique the peptide and nucleic acid components of the desired amino acid and nucleotide sequence can be synthesized separately, e.g. by standard automated chemical synthesis techniques, and then conjugated in aqueous/organic solution. By way of example, the OPeC™ system commercially available from Glen Research is based on the native ligation of an N-terminal thioester-functionalized peptide to a 5′-cysteinyl oligonucleotide.

In some embodiments, a linker can be a “sequencing adaptor” or “sequencing adaptor site”, that is to say a region that comprises one or more sites that can hybridize to a primer. In some embodiments, a linker comprises at least a first primer site. In some embodiments, a linker comprises at least a first primer site and a second primer site. The orientation of the primer sites in such embodiments can be such that a primer hybridizing to the first primer site and a primer hybridizing to the second primer site are in the same orientation, or in different orientations. In one embodiment, the primer sequence in the linker can be complementary to a primer used for amplification. In another embodiment, the primer sequence is complementary to a primer used for sequencing.

In some embodiments, a linker can include a first primer site, a second primer site having a non-amplifiable site disposed therebetween. The non-amplifiable site is useful to block extension of a polynucleotide strand between the first and second primer sites, wherein the polynucleotide strand hybridizes to one of the primer sites. The non-amplifiable site can also be useful to prevent concatamers. Examples of non-amplifiable sites include a nucleotide analogue, non-nucleotide chemical moiety, amino-acid, peptide, and polypeptide. In some embodiments, a non-amplifiable site comprises a nucleotide analogue that does not significantly basepair with A, C, G or T.

Some embodiments include a linker comprising a first primer site, a second primer site having a fragmentation site disposed therebetween.

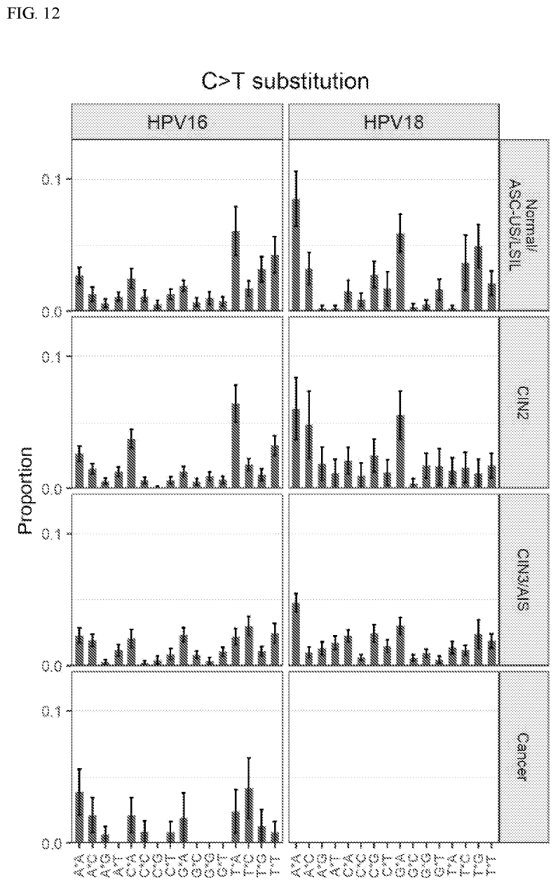

Other embodiments can use a forked or Y-shaped adapter design useful for directional sequencing, as described in U.S. Pat. No. 7,741,463, which is incorporated herein by reference, An example is shown in FIG. 12.

In some embodiments, a linker can comprise an affinity tag. Affinity tags can be useful for the bulk separation of target nucleic acids hybridized to hybridization tags. As used herein, the term “affinity tag” and grammatical equivalents can refer to a component of a multi-component complex, wherein the components of the multi-component complex specifically interact with or bind to each other. For example an affinity tag can include biotin or His that can bind streptavidin or nickel, respectively. Other examples of multiple-component affinity tag complexes include, ligands and their receptors, for example, avidin-biotin, streptavidin-biotin, and derivatives of biotin, streptavidin, or avidin, including, but not limited to, 2-iminobiotin, desthiobiotin, NeutrAvidin (Molecular Probes, Eugene, Oreg.), CaptAvidin (Molecular Probes), and the like; binding proteins/peptides, including maltose-maltose binding protein (MBP), calcium-calcium binding protein/peptide (CBP); antigen-antibody, including epitope tags and their corresponding anti-epitope antibodies; haptens, for example, dinitrophenyl and digoxigenin, and their corresponding antibodies; aptamers and their corresponding targets; poly-His tags (e.g., penta-His and hexa-His) and their binding partners including corresponding immobilized metal ion affinity chromatography (IMAC) materials and anti-poly-His antibodies; fluorophores and anti-fluorophore antibodies; and the like.

A target nucleic acid can include any nucleic acid of interest. Target nucleic acids can include, DNA, RNA, peptide nucleic acid, morpholino nucleic acid, locked nucleic acid, glycol nucleic acid, threose nucleic acid, mixtures thereof, and hybrids thereof. In a preferred embodiment, genomic DNA fragments or amplified copies thereof are used as the target nucleic acid. In another preferred embodiment, mitochondrial or chloroplast DNA is used. As mentioned above, in some preferred embodiments, the target sequence may preferably be from about 1000 to 20000 bases in length, and more preferably from about 3000 to 12000 bases in length. In some particularly preferred embodiments, the target nucleic acid sequence is a sequence that has been inserted or integrated into genomic DNA, for example an integrated viral sequence such as an HPV or HIV sequence.

Some embodiments described herein can utilize a single target nucleic acid. Other embodiments can utilize a plurality of target nucleic acids. In such embodiments, a plurality of target nucleic acids can include a plurality of the same target nucleic acids, a plurality of different target nucleic acids where some target nucleic acids are the same, or a plurality of target nucleic acids where all target nucleic acids are different. Embodiments that utilize a plurality of target nucleic acids can be carried out in multiplex formats such that reagents are delivered simultaneously to the target nucleic acids, for example, in a one or more chambers or on an array surface. In some embodiments, the plurality of target nucleic acids can include substantially all of a particular organism's genome. The plurality of target nucleic acids can include at least a portion of a particular organism's genome including, for example, at least about 1%, 5%, 10%, 25%, 50%, 75%, 80%, 85%, 90%, 95%, or 99% of the genome. In particular embodiments the portion can have an upper limit that is at most about 1%, 5%, 10%, 25%, 50%, 75%, 80%, 85%, 90%, 95%, or 99% of the genome.

Target nucleic acids can be obtained from any source. For example, target nucleic acids may be prepared from nucleic acid molecules obtained from a single organism or from populations of nucleic acid molecules obtained from natural sources that include one or more organisms. Sources of nucleic acid molecules include, but are not limited to, organelles, cells, tissues, organs, or organisms. Cells that may be used as sources of target nucleic acid molecules may be prokaryotic (bacterial cells, for example, Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces genera); archeaon, such as crenarchaeota, nanoarchaeota or euryarchaeotia; or eukaryotic such as fungi, (for example, yeasts), plants, protozoans and other parasites, and animals (including insects (for example, Drosophila spp.), nematodes (for example, Caenorhabditis elegans), and mammals (for example, rat, mouse, monkey, non-human primate and human)).

Some embodiments include methods of preparing template nucleic acids. As used herein, the term “template nucleic acid” can refer to a target nucleic acid, a fragment thereof, or any copy thereof comprising at least one transposon sequence, a fragment thereof, or any copy thereof. Accordingly, some methods of preparing template nucleic acids include inserting a transposon sequence into a target nucleic acid, thereby preparing a template nucleic acid. Some methods of insertion include contacting a transposon sequence provided herein with a target nucleic acid in the presence of an enzyme, such as a transposase or integrase, under conditions sufficient for the integration of the transposon sequence into the target nucleic acid. In some embodiments, the transposon and target nucleic are bound to beads.

Exemplary transposition systems that may be utilized with the compositions and methods provided herein include a hyperactive Tn5 transposase and a Tn5-type transposase recognition site (Goryshin, I. and Reznikoff, W. S., J. Biol. Chem., 273: 7367, 1998; US Pub. 2010/0120098, which is incorporated herein by reference), and MuA transposase and a Mu transposase recognition site comprising R1 and R2 end sequences (Mizuuchi, K., Cell, 35: 785, 1983; Savilahti, H, et al., EMBO J., 14: 4893, 1995). More examples include sequences and enzymes related to Staphylococcus aureus Tn552 (Colegio O R et al., J. Bacteriol., 183: 2384-8, 2001; Kirby C et al., Mol. Microbiol., 43: 173-86, 2002), Ty1 (Devine S E, and Boeke J D., Nucleic Acids Res., 22: 3765-72, 1994 and International Patent Application No. WO 95/23875), Transposon Tn7 (Craig, N L, Science. 271: 1512, 1996; Craig, N L, Review in: Curr Top Microbiol Immunol., 204: 27-48, 1996), Tn/O and IS10 (Kleckner N, et al., Curr Top Microbiol Immunol., 204: 49-82, 1996), Mariner transposase (Lampe D J, et al., EMBO J., 15: 5470-9, 1996), Tc1 (Plasterk R H, Curr Top Microbiol Immunol, 204: 125-43, 1996), P Element (Gloor, G B, Methods Mol. Biol., 260: 97-114, 2004), Tn3 (Ichikawa H, and Ohtsubo E., J. Biol. Chem. 265: 18829-32, 1990), bacterial insertion sequences (Ohtsubo, F and Sekine, Y, Curr. Top. Microbiol. Immunol. 204: 1-26, 1996), retroviruses (Brown P O, et al., Proc Natl Acad Sci USA, 86: 2525-9, 1989), and retrotransposon of yeast (Boeke J D and Corces V G, Annu Rev Microbiol. 43: 403-34, 1989).

In some embodiments, insertion of transposon sequences into a target nucleic acid can be non-random. In some embodiments, transposon sequences can be contacted with target nucleic acids comprising proteins that inhibit integration at certain sites. For example, transposon sequences can be inhibited from integrating into genomic DNA comprising proteins, genomic DNA comprising chromatin, genomic DNA comprising nucleosomes, or genomic DNA comprising histones.

In some embodiments, a plurality of the transposon sequences provided herein is inserted into a target nucleic acid. Some embodiments include selecting conditions sufficient to achieve integration of a plurality of transposon sequences into a target nucleic acid such that the average distance between each integrated transposon sequence comprises a certain number of consecutive nucleotides in the target nucleic acid.

In some embodiments, conditions may be selected so that the average distance in a target nucleic acid between integrated transposon sequences is at least about 5, 10, 20, 30, 40, 50, 60, 70, 80, 90, 100, or more consecutive nucleotides. In some embodiments, the average distance in a target nucleic acid between integrated transposon sequences is at least about 100, 200, 300, 400, 500, 600, 700, 800, 900, 1000, or more consecutive nucleotides. In some embodiments, the average distance in a target nucleic acid between integrated transposon sequences is at least about 1 kb, 2 kb, 3 kb, 4 kb, 5 kb, 6 kb, 7 kb, 8 kb, 90 kb, 100 kb, or more consecutive nucleotides. In some embodiments, the average distance in a target nucleic acid between integrated transposon sequences is at least about 100 kb, 200 kb, 300 kb, 400 kb, 500 kb, 600 kb, 700 kb, 800 kb, 900 kb, 1000 kb, or more consecutive nucleotides. As will be understood, some conditions that may be selected include contacting a target nucleic acid with a certain number of transposon sequences.

Some embodiments include selecting conditions sufficient to achieve at least a portion of transposon sequences integrated into a target nucleic acid are different. In preferred embodiments, each transposon sequence integrated into a target nucleic acid is different. Some conditions that may be selected to achieve a certain portion of transposon sequences integrated into a target sequences that are different include selecting the degree of diversity of the population of transposon sequences. As will be understood, the diversity of transposon sequences arises in part due to the diversity of the barcodes of such transposon sequences. Accordingly, some embodiments include providing a population of transposon sequences in which at least a portion of the barcodes are different. In some embodiments, at least about 10%, 20%, 30%, 40%, 50%, 60%, 70%, 80%, 90%, 95%, 98%, 99%, or 100% of barcodes in a population of transposon sequences are different.

Some embodiments of preparing a template nucleic acid can include copying the sequences comprising the target nucleic acid. For example, some embodiments include hybridizing a primer to a primer site of a transposon sequence integrated into the target nucleic acid. In some such embodiments, the primer can be hybridized to the primer site and extended. The copied sequences can include at least one barcode sequence and at least a portion of the target nucleic acid. In some embodiments, the copied sequences can include a first barcode sequence, a second barcode sequence, and at least a portion of a target nucleic acid disposed therebetween. In some embodiments, at least one copied nucleic acid can include at least a first barcode sequence of a first copied nucleic acid that can be identified or designated to be paired with a second barcode sequence of a second copied nucleic acid. In some embodiments, the primer can include a sequencing primer. In some embodiments sequencing data is obtained using the sequencing primer.

Some embodiments of preparing a template nucleic acid can include amplifying sequences comprising at least a portion of one or more transposon sequences and at least a portion of a target nucleic acid. In some embodiments, at least a portion of a target nucleic acid can be amplified using primers that hybridize to primer sites of integrated transposon sequences integrated into a target nucleic acid. In some such embodiments, an amplified nucleic acid can include a first barcode sequence, and second barcode sequence having at least a portion of the target nucleic acid disposed therebetween. In some embodiments, at least one amplified nucleic acid can include at least a first barcode sequence of a first amplified nucleic acid that can be identified to be paired with a second barcode sequence of a second amplified sequence.

Some embodiments of preparing a template nucleic acid can include fragmenting a target nucleic acid comprising transposon sequences. Methods of fragmenting nucleic acids are well known in the art. In some embodiments, a nucleic acid comprising transposon sequences can be fragmented at random positions along the length of the nucleic acid. In some embodiments, a target nucleic acid comprising transposon sequences can be fragmented at the fragmentation sites of the transposon sequences.

Further embodiments of preparing a template nucleic acid that include fragmenting a target nucleic acid comprising transposon sequences can also include amplifying the fragmented nucleic acids. In some embodiments, the fragmented nucleic acids can be amplified using primers that hybridize to primer sites of transposon sequences. In more embodiments, primer sites can be ligated to the ends of the fragmented nucleic acids. In some such embodiments, the fragmented nucleic acids with ligated primer sites can be amplified from such primer sites.

Some embodiments include reducing the complexity of a library of template nucleic acids. A complexity-reduction step can be performed before or after the fragmentation step in the method. For example, the target nucleic acid comprising the transposon sequences can be diluted so that a small number or a single molecule represents the target diluted before performing subsequent steps.

Some embodiments include methods of analyzing template nucleic acids. Sequencing information can be obtained from a template nucleic acids and a sequence representation of the target nucleic acid can be obtained from such sequencing data.

In some embodiments, a linked read strategy may be used. A linked read strategy can include identifying sequencing data that links at least two sequencing reads. For example, a first sequencing read may contain a first marker, and a second sequencing read may contain a second marker. The first and second markers can identify the sequencing data from each sequencing read to be adjacent in a sequence representation of the target nucleic acid. In some embodiments, markers can comprise a first barcode sequence and a second barcode sequence in which the first barcode sequence can be paired with the second barcode sequence. In more embodiments, markers can comprise a first host tag and a second host tag. In more embodiments, markers can comprise a first barcode sequence with a first host tag, and a second barcode sequence with a second host tag.

An exemplary embodiment of a method for sequencing a template nucleic acid can comprise the following steps. First, sequence the first barcode sequence using a primer hybridizing to the first primer site as the sequencing primer; second, sequence the second barcode sequence using a primer hybridizing to the second primer site as the sequencing primer. The result is two sequence reads that help link the read to its genomic neighbors. Given long enough reads, and short enough library fragments, these two reads can be merged informatically to make one long read that covers the entire fragment. Using the barcode sequence reads and the 9 nucleotide duplicated sequence present from the insertion, reads can now be linked to their genomic neighbors to form much longer “linked reads” in silico.

The methods described herein can be used in conjunction with a variety of sequencing techniques. In some embodiments, the process to determine the nucleotide sequence of a target nucleic acid can be an automated process.

Some embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons.

In another example type of SBS, cycle sequencing is accomplished by stepwise addition of reversible terminator nucleotides containing, for example, a cleavable or photobleachable dye label as described, for example, in U.S. Pat. No. 7,427,67, U.S. Pat. No. 7,414,1163 and U.S. Pat. No. 7,057,026, the disclosures of which are incorporated herein by reference. This approach is being commercialized by Solexa (now Illumina Inc.), and is also described in WO 91/06678 and WO 07/123,744 (filed in the United States patent and trademark Office as U.S. Ser. No. 12/295,337), each of which is incorporated herein by reference in their entireties. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Additional example SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199 and PCT Publication No. WO 07/010,251, the disclosures of which are incorporated herein by reference in their entireties.

Some embodiments can utilize sequencing by ligation techniques. Such techniques utilize DNA ligase to incorporate nucleotides and identify the incorporation of such nucleotides. Example SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can include techniques such as next-next technologies. One example can include nanopore sequencing techniques (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as .alpha.-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). In some such embodiments, nanopore sequencing techniques can be useful to confirm sequence information generated by the methods described herein.

Some embodiments can utilize methods involving the real-time monitoring of DNA polymerase activity. Nucleotide incorporations can be detected through fluorescence resonance energy transfer (FRET) interactions between a fluorophore-bearing polymerase and .gamma.-phosphate-labeled nucleotides as described, for example, in U.S. Pat. Nos. 7,329,492 and 7,211,414 (each of which is incorporated herein by reference in their entireties) or nucleotide incorporations can be detected with zero-mode waveguides as described, for example, in U.S. Pat. No. 7,315,019 (which is incorporated herein by reference in its entirety) and using fluorescent nucleotide analogs and engineered polymerases as described, for example, in U.S. Pat. No. 7,405,281 and U.S. Patent Application Publication No. 2008/0108082 (each of which is incorporated herein by reference in their entireties). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nanostructures.” Proc. Natl. Acad. Sci. USA 105, 1176-1181 (2008), the disclosures of which are incorporated herein by reference in their entireties). In one example single molecule, real-time (SMRT) DNA sequencing technology provided by Pacific Biosciences Inc can be utilized with the methods described herein. In some embodiments, a SMRT chip or the like may be utilized (U.S. Pat. Nos. 7,181,122, 7,302,146, 7,313,308, incorporated by reference in their entireties). A SMRT chip comprises a plurality of zero-mode waveguides (ZMW). Each ZMW comprises a cylindrical hole tens of nanometers in diameter perforating a thin metal film supported by a transparent substrate. When the ZMW is illuminated through the transparent substrate, attenuated light may penetrate the lower 20-30 nm of each ZMW creating a detection volume of about 1×10² L. Smaller detection volumes increase the sensitivity of detecting fluorescent signals by reducing the amount of background that can be observed.

SMRT chips and similar technology can be used in association with nucleotide monomers fluorescently labeled on the terminal phosphate of the nucleotide (Korlach J. et al., “Long, processive enzymatic DNA synthesis using 100% dye-labeled terminal phosphate-linked nucleotides.” Nucleosides, Nucleotides and Nucleic Acids, 27:1072-1083, 2008; incorporated by reference in its entirety). The label is cleaved from the nucleotide monomer on incorporation of the nucleotide into the polynucleotide. Accordingly, the label is not incorporated into the polynucleotide, increasing the signal:background ratio. Moreover, the need for conditions to cleave a label from a labeled nucleotide monomers is reduced.

An additional example of a sequencing platform that may be used in association with some of the embodiments described herein is provided by Helicos Biosciences Corp. In some embodiments, TRUE SINGLE MOLECULE SEQUENCING can be utilized (Harris T. D. et al., “Single Molecule DNA Sequencing of a viral Genome” Science 320:106-109 (2008), incorporated by reference in its entirety). In one embodiment, a library of target nucleic acids can be prepared by the addition of a 3′ poly(A) tail to each target nucleic acid. The poly(A) tail hybridizes to poly(T) oligonucleotides anchored on a glass cover slip. The poly(T) oligonucleotide can be used as a primer for the extension of a polynucleotide complementary to the target nucleic acid. In one embodiment, fluorescently-labeled nucleotide monomer, namely, A, C, G, or T, are delivered one at a time to the target nucleic acid in the presence DNA polymerase. Incorporation of a labeled nucleotide into the polynucleotide complementary to the target nucleic acid is detected, and the position of the fluorescent signal on the glass cover slip indicates the molecule that has been extended. The fluorescent label is removed before the next nucleotide is added to continue the sequencing cycle. Tracking nucleotide incorporation in each polynucleotide strand can provide sequence information for each individual target nucleic acid.

An additional example of a sequencing platform that can be used in association with the methods described herein is provided by Complete Genomics Inc. Libraries of target nucleic acids can be prepared where target nucleic acid sequences are interspersed approximately every 20 bp with adaptor sequences. The target nucleic acids can be amplified using rolling circle replication, and the amplified target nucleic acids can be used to prepare an array of target nucleic acids. Methods of sequencing such arrays include sequencing by ligation, in particular, sequencing by combinatorial probe-anchor ligation (cPAL).

In some embodiments using cPAL, about 10 contiguous bases adjacent to an adaptor may be determined. A pool of probes that includes four distinct labels for each base (A, C, T, G) is used to read the positions adjacent to each adaptor. A separate pool is used to read each position. A pool of probes and an anchor specific to a particular adaptor is delivered to the target nucleic acid in the presence of ligase. The anchor hybridizes to the adaptor, and a probe hybridizes to the target nucleic acid adjacent to the adaptor. The anchor and probe are ligated to one another. The hybridization is detected and the anchor-probe complex is removed. A different anchor and pool of probes is delivered to the target nucleic acid in the presence of ligase.

The sequencing methods described herein can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically coupled to a surface in a spatially distinguishable manner. For example, the target nucleic acids can be bound by direct covalent attachment, attachment to a bead or other particle or associated with a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail herein.

In some embodiments, the nucleic acid template provided herein can be attached to a solid support (“substrate”). Substrates can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

Various methods can be used to attach, anchor or immobilize nucleic acids to the surface of the substrate. The immobilization can be achieved through direct or indirect bonding to the surface. The bonding can be by covalent linkage. See, Joos et al. (1997) Analytical Biochemistry, 247:96-101; Oroskar et al. (1996) Clin. Chem., 42:1547-1555; and Khandjian (1986) Mol. Bio. Rep., 11:107-11. A preferred attachment is direct amine bonding of a terminal nucleotide of the template or the primer to an epoxide integrated on the surface. The bonding also can be through non-covalent linkage. For example, biotin-streptavidin (Taylor et al. (1991) J. Phys. D: Appl. Phys., 24:1443) and digoxigenin with anti-digoxigenin (Smith et al. (1992) Science, 253:1122, are common tools for anchoring nucleic acids to surfaces and parallels. Alternatively, the attachment can be achieved by anchoring a hydrophobic chain into a lipid monolayer or bilayer. Other methods known in the art for attaching nucleic acid molecules to substrates can also be used.

### EXAMPLES

### Example 1

In order to contribute to the understanding of the role of intra-host HPV genomic variability and chromosomal integration in carcinogenesis, we have developed an innovative library preparation strategy followed by an in-house bioinformatics pipeline named TaME-seq (tagmentation-assisted multiplex PCR enrichment sequencing). TaME-seq combines tagmentation and multiplex PCR enrichment, allowing simultaneous HPV genomic variability and integration analysis (FIG. 1). TaME-seq, with highly efficient target enrichment and reduced sequencing cost, enables deep sequencing analysis in order to find low frequency variants and rare integration events. Here, we present the results of HPV integration and genomic variability analysis in HPV16, 18, 31, 33 and 45 positive clinical samples and cell lines. The method described here provides an important tool for comprehensive studies of HPV genomic variability and chromosomal integration, and it can also be adapted to studies on other viruses such as retroviruses, adeno-associated viruses and integrating human herpesviruses.

### Methods

Samples. Anonymised liquid-based cytology (LBC) samples from routine cervical cancer screening were included in the study, comprising cases of atypical squamous cells of undetermined significance (ASC-US) and low-grade squamous intraepithelial lesions (LSIL). HPV positive samples with the cobas 4800 HPV test (Roche Molecular Diagnostics, Pleasanton, Calif.) were extracted for DNA using the automated system NucliSENS easyMAG (BioMerieux Inc., France) with off-board lysis. The samples were HPV genotyped using the modified GP5+/6+ PCR protocol (MGP)⁵², followed by HPV type-specific hybridisation using Luminex suspension array technology⁵³ or the Anyplex™ II HIPV28 assay (Seegene, Inc., Seoul, Korea). LBC samples (n=31) were positive for HPV16, 18, 31, 33 or 45 alone, or had multiple infections including at least one of the five types. DNA extracted from the HPV positive cervical carcinoma cell lines CaSki, SiHa, HeLa and MS751 (ATCC, Manassas, Va.) served as positive controls. WHO international standards for HPV 16 (1st WHO International Standard for Human Papillomavirus Type 16 DNA, NIBSC code: 06/202) and 18 (1st WHO International Standard for Human Papillomavirus Type 18 DNA, NIBSC code: 06/206)(NIBSC, Potters Bar, Hertfordshire, UK) and a plasmid containing the strain HPV33⁵⁴ were used as additional positive controls. Laboratory-grade water and DNA from an HPV negative human sample were included as negative controls. DNA was quantified by the fluorescence-based Qubit dsDNA HS assay (Thermo Fisher Scientific Inc., Waltham, Mass., USA).

Primer design. HPV16, 18, 31, 33, and 45 whole genome reference and variant sequences were obtained from the PapillomaVirus Episteme (PaVE) database⁵⁵. All the available reference and variant sequences within an HPV type were aligned using the multiple sequence alignment tool ClustalO⁵⁶. The sequence alignment was converted to a consensus sequence for each HPV type in CLC Sequence viewer version 7.7.1 (QIAGEN Aarhus A/S). TaME-seq HPV primers were designed using Primer3⁵⁷ and HPV consensus sequences as the source sequence. Finally, primers were modified by adding an Illumina TruSeq-compatible adapter tail (5′-AGACGTGTGCTCTTCCGATCT-3′(SEQ ID NO: 3)) to the 5′-end and then synthesised by Thermo Fisher Scientific, Inc. (Waltham, Mass.).

Library preparation and sequencing. Primer pools for each HPV type were prepared by combining primers separately in equal volumes. Samples were subjected to tagmentation using Nextera DNA library prep kit (Illumina, Inc., San Diego, Calif.). Tagmented DNA was purified using DNA Clean & Concentrator™-5 columns (Zymo Research, Irvine, Calif.) according the manufacturer's instructions or ZR-96 DNA Clean & Concentrator™-5 plates (Zymo Research, Irvine, Calif.) according to the Nextera® DNA Library Prep Reference Guide (15027987 v01) before PCR amplification for target enrichment. Amplification was performed using Qiagen Multiplex PCR Master mix (Qiagen, Hilden, Germany) according to the manufacturer's instructions. For each sample, two PCR reactions were performed separately with 0.75 μM of HPV primer pools, 0.5 μM of i7 index primers (adapted from Kozich et al., 2013⁵⁸) and 1 μl of i5 index primers from the Nextera index kit (Illumina, Inc., San Diego, Calif.). The cycling conditions were as follows: initial denaturation and hot start at 95° C. for 5 minutes; 30 cycles at 95° C. for 30 seconds, at 58° C. for 90 seconds and at 72° C. for 20 seconds; final extension at 68° C. for 10 minutes. Following amplification, libraries were pooled in equal volumes and the final sample pool was purified with Agencourt© AMPure® XP beads (Beckman Coulter, Brea, Calif.). The quality and quantity of the pooled libraries were assessed on Agilent 2100 Bioanalyzer using Agilent High Sensitivity DNA Kit (Agilent Technologies Inc., Santa Clara, Calif.) and by qPCR using KAPA DNA library quantification kit (Kapa Biosystems, Wilmington, Mass.). Sequencing was performed on the MiSeqplatform (Illumina, Inc., San Diego, Calif.) or on the HiSeq 2500 platform (Illumina, Inc., San Diego, Calif.). Samples were sequenced as 151 bp paired-end reads and two 8 bp index reads.

Sequence alignment. Raw paired-end reads were trimmed for adapters, HPV primers, quality (−q 20) and finally for minimum length (-m 50) using cutadapt (v1.10)⁵⁹. Trimmed reads were mapped to human (GRCh38/hg38) and HPV16, 18, 31, 33 and 45 reference genomes obtained from the PaVE database⁵⁵ using HISAT2 (v2.1.0)⁶⁰. Mapping statistics and sequencing coverage were calculated using the Pysam package⁶¹ with an in-house Python (v3.5.4) script. Downstream analysis was performed using an in-house R (v3.4.4) script. Results from both reactions of the same sample were combined and method performance was then evaluated based on the percentage of obtained reads mapped to the HPV reference genome, mean sequencing coverage and percentage of HPV reference genome coverage for each sample. Further analysis was performed when a sample had >20000 reads mapped to the target HPV reference genome. The target HPV genomes correspond to the HPV types for which the samples were reported positive by HPV genotyping.

Detecting HPV-human integration sites. The paired-end reads that mapped (HISAT2) with one end to a human chromosome and the other end to the target HPV reference genome were identified as discordant read pairs. If a specific position had ≥2 read pairs with unique start or end coordinates, it was considered as a potential integration site. To determine the exact position of HPV-human integration breakpoints, previously unmapped reads were re-mapped to human and HPV reference genomes (as above) using the LAST (v876) aligner (options -M-C2)⁶². Positions covered by ≥3 junction reads, with unique start or end coordinates, were considered as potential integration breakpoints. Integration site detection was not based on reads sharing the same start and end coordinates as these reads were considered as potential PCR duplicates. Selected HPV integration breakpoints were confirmed by PCR amplification and Sanger sequencing.

Sequence variation analysis. Mapped nucleotide counts over HPV reference genomes and average mapping quality values of each nucleotide were retrieved from BAM files and variant calling was performed using an in-house R script. To reduce the effects of PCR amplification and sequencing artefacts in the variation analysis, filtering was applied before the variant calling. Nucleotides seen ≤2 times in each position and nucleotides with mean Phred quality score of <20 were filtered out. Nucleotide counts from both reactions of the same sample were combined and variant allele frequencies (VAF) of the three minor alleles in each position were calculated. If results from either of the reaction showed >5 times larger VAF with <20% of the total coverage, it was discarded from variant calling. Finally, variants were called if VAF was >0.2% and coverage was ≥100×.

Two sequencing libraries of SiHa cell line served as technical replicates to assess the variant calling performance. The technical replicates were sequenced on the MiSeqplatform or on the HiSeq 2500 platform. In addition, HiSeq raw sequencing data was downsampled randomly and defined portions (90%, 75%, 50% and 25%) of the original reads were further analysed. Reproducibility of calling variants in the replicates was assessed by calculating concordance rate. The concordance rate (Re) between duplicates was defined as follows:

$${R}_{c} = \frac{{N}_{c}}{mean ( {N}_{1} , {N}_{2} )}$$

where N_{c} was the number of concordant variants between a pair of replicate samples, and N₁ and N₂ were the total number of variants detected in each of the duplicated sample.

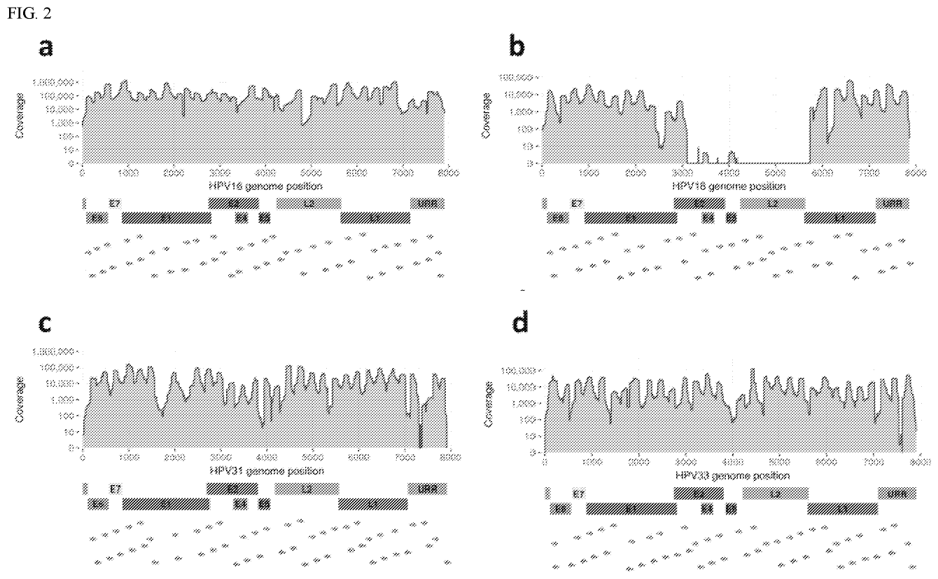

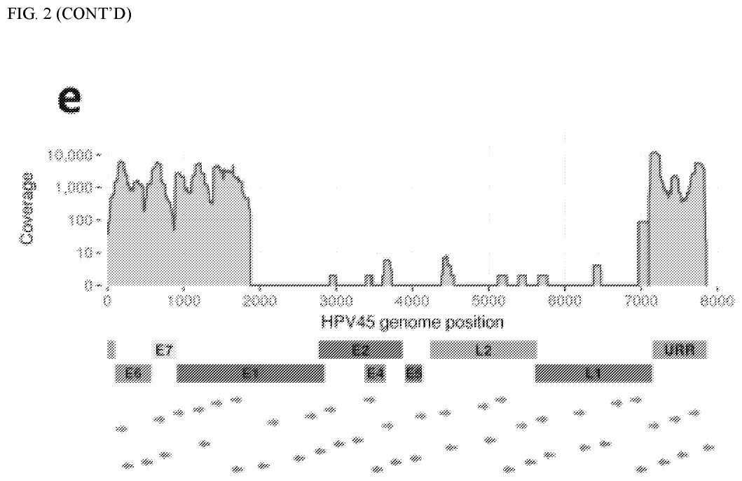

Results Read mapping analysis and genome coverage. Table 1 summarises LBC samples (n=21), cell lines (n=4) and plasmid samples (n=3) included in the analysis. The samples generated 154.8 million raw reads of which 72.5 million reads (47%) mapped to the target HPV reference genomes. Only a small fraction (0.08%) of the reads mapped to other HPV types than those reported positive by HPV genotyping. The mean coverage ranged from 303 to 273898, while the fraction of the genome covered by minimum 10× ranged from 0.35 to 1, and the fraction of the genome covered by minimum 100× ranged from 0.33 to 1 (Table 1). HPV genome sequencing coverage aligned to the target HPV genomes with the location of HPV genomic regions and primers is visualised for CaSki, HeLa, LBC34, LBC11 and MS751 (FIG. 2). Overall, the samples showed varying HPV genome coverage profiles (data not shown). Totally, 10 HPV positive samples were excluded from further analysis due to poor sequencing coverage (data not shown). Sequencing of the HPV negative control samples resulted in no or negligible amount (<500) of reads mapped to target HPV genomes (data not shown). The MS751 cell line was confirmed not to contain HPV18 sequences (data not shown)³⁵.

TABLE 1
Read counts and sequencing coverage of HPV positive cell lines, plasmids and LBC samples.

| | | | | Reads | % Reads | | Fraction of | |
|---|---|---|---|---|---|---|---|---|
| | | | | mapped | mapped | | genome covered | |
| | Sample | Raw | Trimmed | to target | to target | Mean | by minimum | |
| Sample | type | reads | reads | HPV | HPV | coverage | 10× | 100× |
| HPV16 | | | | | | | | |
| CaSki | Cell line | 16138790^{b} | 12944262 | 12634651 | 78% | 184716 | 1.00 | 1.00 |
| SiHa | Cell line | 151168^{b} | 133360 | 67496 | 45% | 1018 | 0.96 | 0.83 |
| SiHa-1 | Cell line | 5948008^{c} | 3735936 | 1249594 | 21% | 17561 | 0.93 | 0.90 |
| SiHa-1 | Cell line | 844178^{b} | 532874 | 181199 | 21% | 2554 | 0.92 | 0.78 |
| SiHa-2 | Cell line | 1405886^{c} | 789664 | 420774 | 30% | 5609 | 0.91 | 0.85 |
| SiHa-2 | Cell line | 158672^{b} | 90150 | 48412 | 31% | 646 | 0.84 | 0.52 |
| WHO std HPV16 | Plasmid | 359638^{b} | 304002 | 278987 | 78% | 4104 | 0.99 | 0.96 |
| LBCl^{a} | LBC | 128008^{b} | 108756 | 75323 | 59% | 1124 | 0.96 | 0.88 |
| LBC7^{a} | LBC | 62246^{b} | 51590 | 25567 | 41% | 384 | 0.94 | 0.66 |
| HPV18 | | | | | | | | |
| HeLa | Cell line | 1433248^{b} | 1120824 | 394420 | 28% | 5897 | 0.68 | 0.62 |
| WHO std HPV18 | Plasmid | 2021206^{b} | 1358182 | 1098783 | 54% | 15447 | 0.99 | 0.96 |
| LBC103^{a} | LBC | 1477706^{b} | 1209564 | 74358 | 5% | 1056 | 0.93 | 0.83 |
| LBC105^{a} | LBC | 190664^{b} | 160450 | 32695 | 17% | 484 | 0.51 | 0.34 |
| LBC107 | LBC | 2180284^{b} | 1881868 | 978435 | 45% | 14663 | 1.00 | 0.99 |
| LBC108^{a} | LBC | 5407154^{b} | 3773986 | 3360463 | 62% | 46691 | 1.00 | 0.98 |
| LBC48^{a} | LBC | 641378^{b} | 433884 | 72589 | 11% | 988 | 0.95 | 0.83 |
| HPV31 | | | | | | | | |
| LBC16 | LBC | 276994^{b} | 191290 | 74465 | 27% | 1065 | 0.94 | 0.80 |
| LBC24^{a} | LBC | 471666^{b} | 348416 | 24197 | 5% | 355 | 0.96 | 0.69 |
| LBC32 | LBC | 2446832^{b} | 1523572 | 1319939 | 54% | 18983 | 0.99 | 0.98 |
| LBC34 | LBC | 3285680^{b} | 1841812 | 1723631 | 52% | 23790 | 0.99 | 0.96 |
| HPV33 | | | | | | | | |
| HPV33 plasmid | Plasmid | 13824396^{b} | 5202718 | 5230090 | 38% | 61527 | 1.00 | 1.00 |
| LBC11 | LBC | 2852262^{b} | 1052512 | 986936 | 35% | 12038 | 0.99 | 0.98 |
| LBC30 | LBC | 77128^{b} | 51682 | 21431 | 28% | 303 | 0.93 | 0.63 |
| LBC31^{a} | LBC | 4276740^{c} | 2831408 | 44917 | 1.1% | 544 | 0.76 | 0.60 |
| LBC52 | LBC | 154936^{b} | 86990 | 34390 | 22% | 439 | 0.95 | 0.62 |
| LBC65^{a} | LBC | 368260^{b} | 248142 | 144022 | 39% | 1993 | 1.00 | 0.91 |
| HPV45 | | | | | | | | |
| MS751 | Cell line | 1221694^{b} | 1047286 | 56291 | 5% | 845 | 0.35 | 0.33 |
| LBC13^{a} | LBC | 496370^{b} | 389306 | 58293 | 12% | 849 | 0.96 | 0.78 |
| LBC29 | LBC | 211052^{b} | 122502 | 45925 | 22% | 614 | 0.91 | 0.69 |
| LBC36^{a} | LBC | 2412532^{b} | 1822912 | 1579570 | 65% | 22093 | 1.00 | 0.97 |
| LBC54 | LBC | 50169422^{c} | 26385910 | 20570184 | 41% | 256857 | 1.00 | 1.00 |
| LBC64^{a} | LBC | 5121416^{c} | 3040714 | 307476 | 6% | 3943 | 0.95 | 0.88 |

^{a}Sample has multiple HPV infections.
^{b}Sequenced on MiSeq sequencing platform.
^{c}Sequenced on HiSeq 2500 sequencing platform.

Deletions in HPV genomes. The method enables identification of regions covered with very few or no sequencing reads, interpreted as large HPV genomic deletions. Cell lines HeLa and MS751 are known to contain partial HPV genomes due to deletions of 2.5 kb and 5 kb, respectively^{35,36}, which was confirmed by our method (FIG. 2). A large deletion of 4.8 kb was revealed in the clinical sample LBC105, indicating partial or complete deletion of HPV18 genes E1, E2, E4, E5, L1 and L2 (data not shown).

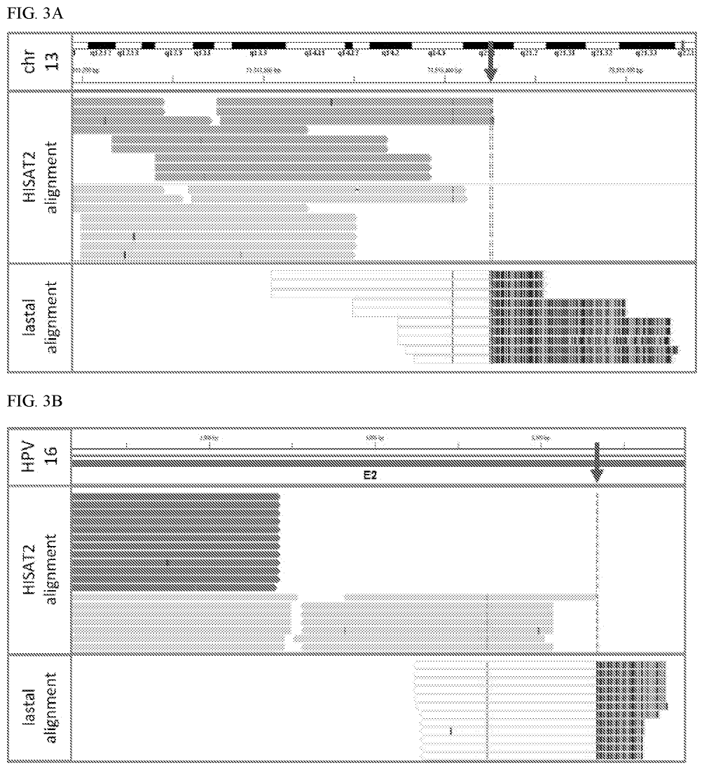

HPV-human integration sites. A two-step strategy was applied to detect possible integration sites (FIG. 3). A total of 27 integration sites were detected in cell lines CaSki, SiHa, HeLa and MS571 (Table 2). For CaSki, 16 previously reported integration sites^{30,32,37} were confirmed. In addition, three novel sites were identified. These mapped to HPV16 E6, E2 and L1 genes. One was located in an intronic region of the gene BRSK1; two were located more than 50 kb from annotated genes (Table 2). Three sites, including one previously reported site as a control^{30,37}, were subjected to Sanger sequencing to confirm the integration sites (data not shown). Integration sites identified in SiHa, HeLa and MS751 were consistent with previous studies^{31,35-39} and were not subjected to validation by Sanger sequencing. Additionally, two integration sites were detected in the clinical sample LBC105 (Table 2). The integration breakpoints were mapped to the HPV E1 and L1 genes flanking the deleted region (data not shown) and they were located in intronic regions of the gene GTF2JRDI (Table 2). Both integration sites were confirmed by Sanger sequencing (data not shown).

TABLE 2
Chromosomal integration sites detected by TaME-seq.

| | | | Human (GRCh38/hg38) | | # Unique | # Unique |
|---|---|---|---|---|---|---|
| | HPV | | Chromosomal | | discordant | junction |
| Sample | Breakpoint | ORF | locus | Breakpoint | read pairs | reads |
| HPV16 | | | | | | |
| CaSki | 273 | E6 | 20p11.1 | chr20: 26276796 | 19 | 0^{e} |
| | 494^{a} | E6 | 20p11.1 | chr20: 26341342^{b} | 7 | 0^{e} |
| | 582 | E7 | 19q13.42 | chr19: 55310208 | 0 | 15 |
| | 975 | E1 | Xq27.3 | chrX: 145696778 | 0 | 7 |
| | 1398 | E1 | 2p23.3 | chr2: 27135968 | 6 | 0^{e} |
| | 1793 | E1 | 10p14 | chr10: 11700197 | 4 | 0^{e} |
| | 2987 | E2 | Xq27.3 | chrX: 145708231 | 3 | 8 |
| | 3239 | E2 | 7p22.1 | chr7: 6925283 | 5 | 0^{e} |
| | 3631^{a} | E2 | 19q13.42 | chr19: 55310043^{c} | 3 | 0^{e} |
| | 3729 | E2 | 6p21.1 | chr6: 45691388 | 0 | 11 |
| | 4654 | L2 | 11p15.4 | chr11: 6741077 | 11 | 0^{e} |
| | 5432 | L2 | 11q22.1 | chr11: 100766632 | 2 | 0^{e} |
| | 5698 | L1 | 10p14 | chr10: 11700617 | 20 | 0^{e} |
| | 5698 | L1 | 5p11 | chr5: 46292081 | 2 | 0^{e} |
| | 5762 | L1 | 11q22.1 | chr11: 100771699 | 4 | 0^{e} |
| | 6572 | L1 | 19q13.42 | chr19: 55307445 | 3 | 0^{e} |
| | 7123^{a} | L1 | 20p11.1 | chr20: 26357640^{b} | 20 | 0^{e} |
| | 7733 | URR | 11p15.4 | chr11: 6740842 | 2 | 0^{e} |
| | 7733 | URR | 2p23.3 | chr2: 27137265 | 6 | 0^{e} |
| SiHa | 3133 | E2 | 13q22.1 | chr13: 73513425 | 7 | 7 |
| | 3385 | E2/E4 | 13q22.1 | chr13: 73214729 | 3 | 0^{e} |
| HPV18 | | | | | | |
| HeLa | 2066 | E1 | 8q24.21 | chr8: 127229053 | 2 | 0^{e} |
| | 2887 | E2 | 8q24.21 | chr8: 127221122 | 13 | 0^{e} |
| | 5730 | L1 | 8q24.21 | chr8: 127218384 | 11 | 89 |
| | 7655 | URR | 8q24.21 | chr8: 127221804 | 3 | 0^{e} |
| LBC105 | 1561 | E1 | 7q11.23 | chr7: 74525628^{d} | 0 | 10 |
| LBC105 | 6528 | L1 | 7q11.23 | chr7: 74515883^{d} | 2 | 0^{e} |
| HPV45 | | | | | | |
| MS751 | 1646 | E1 | 18q11.2 | chr18: 23024744 | 10 | 0^{e} |
| | 7120 | L1 | 18q11.2 | chr18: 23021388 | 15 | 0^{e} |

^{a}Novel breakpoint in CaSki cell line.
^{b}No annotated genes within 50 kB from the breakpoint.
^{c}Intronic region in gene BRSK1.
^{d}Intronic region in gene GTF2IRD1.
^{e}When number of unique junction reads is 0, the breakpoint coordinates are not exact.

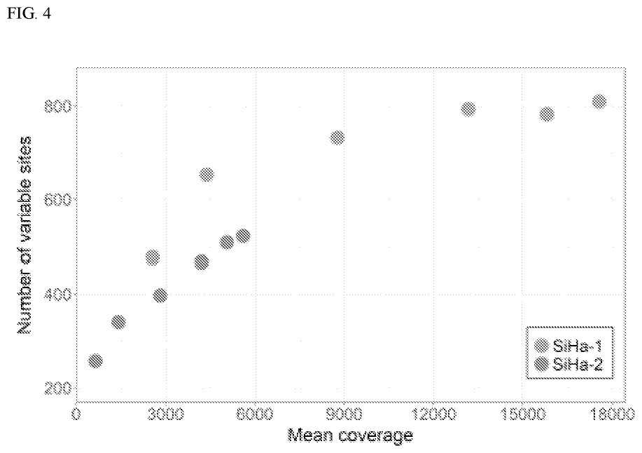

Evaluation of variant calling using SiHa technical replicates. Sequencing libraries of the SiHa cell line served as technical replicates to assess the variant calling performance. In both SiHa-1 and SiHa-2, more variable sites were detected with higher mean coverage (FIG. 4). Number of variable sites in SiHa-1 ranged from 477 to 809 and mean coverage ranged from 2554 to 17561. Number of variable sites in SiHa-2 ranged from 257 to 522 and mean coverage ranged from 646 to 5609 (FIG. 4; data not shown). First, reproducibility of variant calling was assessed within the same SiHa sequencing library. Concordance rate of variable sites was calculated using HiSeq 2500 result as the reference value. The concordance rates varied from 92% (HiSeq downsampled 90%) to 45% (MiSeq) in SiHa-1 and from 89% (HiSeq downsampled 90%) to 27% (MiSeq) in SiHa-2 (data not shown). Concordance rates of variants, including low frequency variation, between replicates (different library, same sequencing platform) were calculated to evaluate the effect of library preparation steps on the number of variable sites found in each sample. Concordance rates were 21% and 19% in SiHa-1 and SiHa-2, respectively (data not shown).

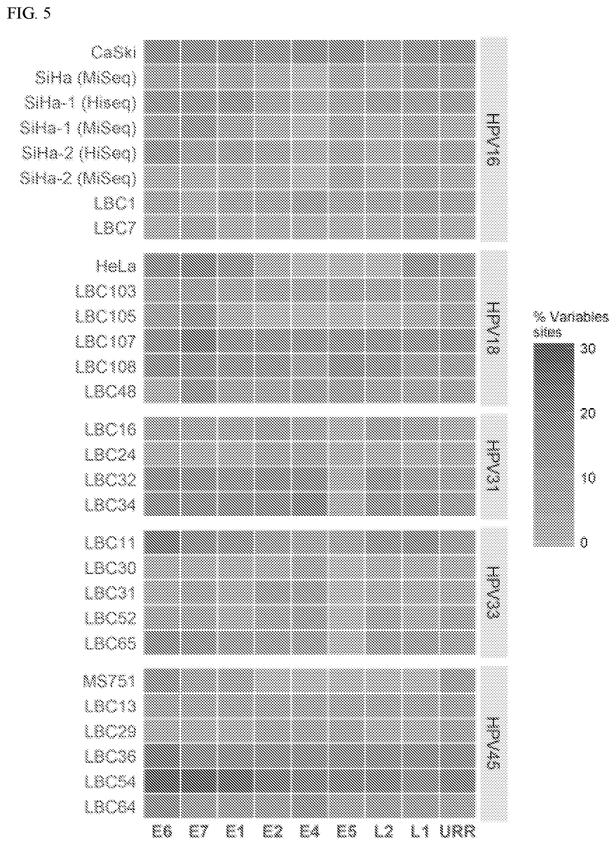

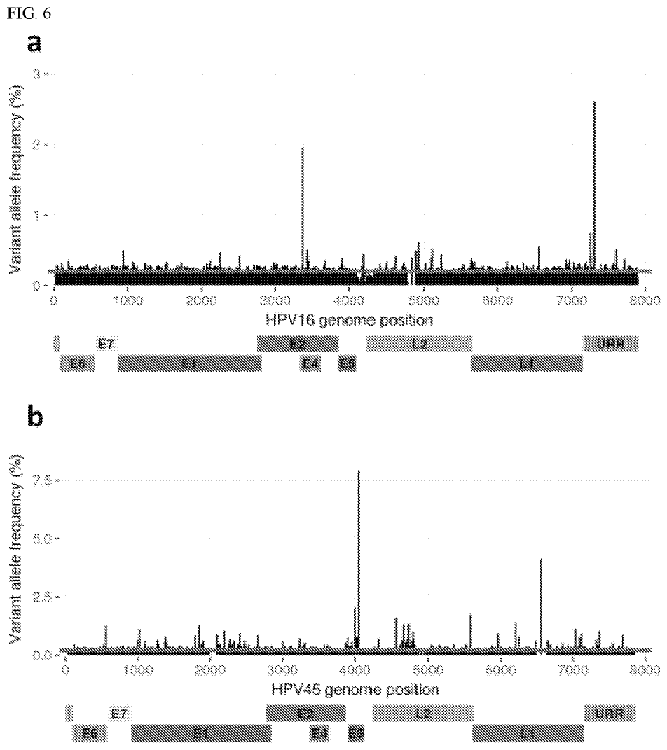

HPV genomic variability. Variability was analysed in cell lines and LBC samples. Samples had variable sites (variant allele frequency >0.2% and coverage ≥100×) in all genes with the exception of regions that were deleted or had low sequencing coverage. The number of variable sites were normalized by the length of each HPV genomic region. Genomic regions had varying percentages of variable sites (0-28%) in each of the samples. Overall, there were samples within each HPV type that had >15% variable sites in at least one HPV gene (FIG. 5). Principally, samples with higher mean coverage had more variable sites (data not shown), which is in line with the results from the variant analysis done on SiHa replicates (FIG. 4). CaSki had most variable sites (1017) of the cell lines and LBC54 had most variable sites (1641) of the clinical samples (data not shown). A variant profile with variable site positions and variant allele frequency (VAF) is shown for CaSki and LBC54 (FIG. 6). Overall, the results show considerable variability in the samples throughout the HPV genome (FIG. 5, data not shown).

Discussion

Here, we present a novel cost-efficient approach, TaME-seq, for the simultaneous analysis of HPV variation and chromosomal integration. Previous methods have been less effective and/or limited to either one of the two analyses²⁹⁻³⁴. To demonstrate the performance of TaME-seq, we employed HPV16, 18, 31, 33 and 45 positive clinical samples, HPV positive cell lines and HPV plasmids. With 47% of the total of 154.8 million raw reads mapped on the target HPV reference genomes, TaME-seq proved to be highly efficient in HPV target enrichment. Other approaches for HPV target enrichment have reported much lower HPV mapping ratios^{32,40} requiring more sequencing and therefore at a higher sequencing cost. TaME-seq currently covers HPV16, 18, 31, 33 and 45, being the most common HPV genotypes in cervical cancer⁵. TaME-seq can be extended to cover additional HPV types, as well as other viruses, by implementing new primers to the method.

The ability of TaME-seq to detect chromosomal integration sites has been shown for the HPV positive cervical cancer cell lines CaSki, SiHa, HeLa and MS751. CaSki cells contain a high copy number (˜600 copies/cell) of integrated full-length HPV16 arranged in concatemers^{41,42}. SiHa (1-2 HIPV16 copies/cell)^{39,41} and HeLa (10-50 HPV18 copies/cell)⁴³ cells harbor integrated HPV genomes. MS751 cells contains integrated HPV45³⁵, but in contrast to the product specification sheet (ATCC, Manassas, Va.) no HPV18, which was verified in our analyses. For CaSki, 16 previously reported integration sites^{30,32,37} were detected by our method. In addition, three novel integration sites were identified. Known integration sites in SiHa^{31,37,39}, HeLa^{31,36} and MS751³⁵, as well as large deletions demonstrated in HeLa³⁶ and MS751³⁵, were confirmed by the TaME-seq method. Of the 21 LBC samples, HPV integration sites could only be detected in one sample, being in line with previous studies reporting no or few HPV integration events in LSIL/ASC-US samples^{44,45}. However, other studies report integration events also in LSIL samples^{32,46}. The detection of integrated forms of the virus is also dependent on the amount of episomes in the sample; low copy integration sites may remain undetected against a high background of episomal HPV.

The high sequencing coverage throughout the HPV genome enables detection of low frequency variants. Variant calling was evaluated using SiHa replicates to set the variant calling threshold. Previous studies have used variant calling thresholds of 0.5% or 1%^{17,34} With the high coverage provided by the TaME-seq method there is potential for detecting very low frequency variation. We have therefore analysed the variation using 0.2% as the variant calling threshold. Multiple and stringent filtering steps was included to filter out non-reliable variants, as we are approaching the inherent error rate profile of the PCR amplification and Illumina sequencing⁴⁷. However, the threshold for variant calling is dependent on experimental and analytical basis and must be set according to the study aims.

The results from the SiHa analysis indicate that calling ultra-low frequency variants is dependent on the sequencing coverage. Lower sequencing coverage results in the detection of fewer variants and less concordance between sample replicates. In order to find ultra-low frequency variants, high sequencing coverage is required. FIG. 4 shows that at the mean coverage of 12000×, the number of variants in SiHa-1 is approaching saturation. This indicates that more variants are not likely to be found even with higher sequencing coverage. Finally, differences in sequencing coverage affect the number of variable sites found, but also experimental approaches due to stochastic sampling and variant calling can fail to reveal low frequency variants. Overall, our results uncover low frequency variants in the samples, potentially introduced by DNA repair mechanisms and APOBEC enzyme mediated DNA editing⁴⁸⁻⁵⁰, although some bias may be introduced by PCR and sequencing. Variable sites are present in all genes of the studied HPV types. Traditionally, studies have focused on sequence variation on a viral sublineage level¹³⁻¹⁶ or the high variability has been interpreted as HPV variant co-infections²⁹. The development of NGS technologies has provided comprehensive tools for the study of HPV genomic variability. Recent studies have reported high HPV variability that may be evidence of intra-host viral evolution and adaption generated during a chronic HPV infectioni¹⁷⁻²⁰.

Our study has some limitations. Firstly, TaME-seq is not intended for determining HPV genotypes and we recommend it for analyses of HPV variability and integration events in samples with known HPV status. Secondly, due to variation in amplification efficacy, an uneven coverage is seen for different genomic regions. Sudden drops in the coverage, that are not genomic deletions, may be due to suboptimal primer performance or poor alignment against the reference genomes. This issue can be solved partly by designing new primers covering these regions and optimizing the primer performance. Also, the read alignment step can be further optimized. Alternatively, alignment could be performed by de novo assembly to create consensus sequences for the alignment. Thirdly, enough viral DNA and good dsDNA quality is important for achieving consistent tagmentation results in the Nextera protocol⁵¹. Sample preparation of excluded LBC samples failed likely due to very low viral load in the samples, which was not quantified separately.

In summary, we have developed a NGS approach that allows the simultaneous study of HPV genomic variability and chromosomal integration. TaME-seq is applicable to large sample cohorts due to its highly efficient target enrichment, leading to less off-target sequences and therefore reduced sequencing cost. Comprehensive studies on HPV intra-host variability generated during a persistent infection will improve our understanding of viral carcinogenesis. Efficient identification of HPV genomic variability and integration sites will be important both for the study of HPV evolution, adaptability and may be a useful tool for cervical cancer diagnostics.

### Example 2

Deep sequencing allows for in-depth characterization of HPV events in carcinogenesis, such as the generation of minor nucleotide variants and chromosomal integration events. Recent studies have revealed genomic variability indicating intra-host viral evolution and adaptation acquired through various mutagenic processes, one of which is APOBEC. This example provides a comparison of the extent and nature of genomic events in HPV16 and HPV18 positive clinical samples with different morphology.

Briefly, HPV16 (n=157) and HPV18 (n=75) positive cervical samples were included, categorized into the four categories normal/ASCUS/LSIL with no lesions within four years follow up (n=71), CIN2 (n=60), CIN3/AIS (n=96) and ICC (n=5). Samples were sequenced using the whole genome HPV deep sequencing protocol TaME-seq, assessing both nucleotide variants, viral genomic deletions and chromosomal integration.

Samples with a mean coverage >300× (n=131) were included for analyses. Sequence analyses revealed a higher overall HPV integration rate in HPV18 positive samples compared to HPV16, characterized in 30/51 (59%) of HPV18 positive samples and in 10/80 (13%) of HPV16 positive samples. In addition, the number of integration breakpoints per sample was generally higher for HPV18 compared to HPV16 positive samples, ranging from 1 to 21 integrations per sample. Considering CIN3/AIS/ICC samples showing integration events, 8 of 10 HPV16-human breakpoints (80%; n=4) and 37 of 60 HPV18-human breakpoints (62%; n=14) were located in or in close proximity to cancer-related genes. Similar rates of minor nucleotide variants in HIPV16 and HPV18 sequences were observed, with distinct APOBEC signatures for HPV16.

### Methods

Sample selection. Cervical cell samples were collected from women attending the cervical cancer screening program in Norway between January 2005 and April 2008, included in a research biobank at Akershus University Hospital, consisting of both the cell material and DNA. Recruitment criteria and HPV detection and genotyping have been described previously^{63,64}. Cytological samples from the women were previously analyzed for HPV using the Amplicor HPV DNA test detecting 13 HPV types (Roche Diagnostics, Switzerland) followed by genotyping by Linear Array (Roche Diagnostics, Switzerland) and by PreTect HPV Proofer detecting HPV E6/E7 mRNA from HPV 16, 18, 31, 33 and 45 (NorChip, Norway). Samples were collected in ThinPrep PreservCyt Solution (Hologic) and pelleted before storage at −80° C. In this study, primarily DNA was used for downstream analyses; for some samples, DNA extraction had to be performed from the cell material. Inclusion criteria for this study were the following: Samples positive for HPV 16 and/or 18 alone or together with other HPV types by one or both of the genotyping methods. All HPV16 and/or HPV18 positive samples with normal cytology (n=27) were included. To expand this category, atypical squamous cells of undetermined significance (ASC-US) and low-grade squamous intraepithelial lesions (LSIL) from women with no follow-up diagnosis within four years subsequent to the ASC-US/LSIL diagnosis, were included to a total number of 71 in this category. This category is in this study referred to as the normal group. A random selection of up to 50 cytological samples representing women with histologically confirmed cervical intraepithelial neoplasia (CIN) grade 2 or 3, or adenocarcinoma in situ (ACIS) were included, in addition to cytological samples from women with cervical cancer (including both squamous cell carcinoma and adenocarcinoma). In total, 157 HPV16 positive samples and 75 HPV18 positive samples were subjected to sequencing (Table 3).

Library preparation and sequencing. Library preparation was performed using the TaME-seq method as described previously⁶⁵. In brief, samples were subjected to tagmentation using Nextera DNA library prep kit (Illumina, Inc., San Diego, Calif.), following target enrichment performed by multiplex PCR using HPV primers and a combination of i7 index primers⁶⁶ and i5 index primers from the Nextera index kit (Illumina, Inc., San Diego, Calif.). Sequencing was performed on the HiSeq2500 platform with 125 bp paired-end reads length.

Sequence alignment. Data was analyzed by an in-house bioinformatics pipeline as described previously30. Reads were mapped to human genome (GRCh38/hg38) using HISAT2 (v2.1.0)34. HPV 16 and HPV 18 reference genomes were obtained from the PaVE database⁶⁷. Mapping statistics and sequencing coverage were calculated using the Pysam package⁶⁸ with an in-house Python (v3.5.4) script. Downstream analysis was performed using an in-house R (v3.5.1) script. Samples with a mean coverage of <300× reads were excluded from the further analysis.

Detection of chromosomal integration sites. Integration site detection was performed as described previously⁶⁵. We employed a two-step analysis strategy to identify read pairs spanning integrations sites. First, we identified read pairs with one mapped to HPV and the other to the human chromosome or one of the reads in the pair mapped to human chromosome in one end and HPV in the other using HISAT2. Second, unmapped reads were re-mapped using the LAST (v876) aligner (options -M-C2)⁶⁹ to increase detections of the above mentioned read pairs. Reads sharing the same start and end coordinates were considered potential PCR duplicates and were excluded. Selected integration sites were confirmed by PCR amplification and Sanger sequencing. The data of any sample with a mean coverage of >1000× and <0.85 of the genome covered by >100× were manually inspected for large genomic deletions using IGV (v2.3.09).

Functional annotation of genes in or in close vicinity to integration sites. Nearest gene, with a transcription starting site within 100 kb distance from the integration site were identified using the gene set from Ensembl. Genomic elements annotations (genes, exons/introns, non-coding RNA (ncRNA), antisense RNA, retained introns and UTR) were included if they had transcript support level of 2 or more. Information on regulatory elements were retrieved from Ensembl's regulatory build and included promoters, promoter flanking regions, enhancers and CTCF-binding sites.

Gene2function (ref) and Genecards were used to annotate the function and disease phenotype of each of the nearest genes. Molecular functions of genes as well as SNP associations from the GWAS catalog (Welter et al., 2014) were retrieved from Genecards. Genes belonging to cell cycle regulation, cell proliferation, apoptosis, tumor suppressor mechanisms or cancer-related pathways, or interacted with genes in these pathways, were here termed a cancer-related gene. Annotations surrounding the integration breakpoints were manually inspected using the Geneious Prime (v. 2019.0.4)

Sequence variation analysis. Mapped nucleotide counts over the HPV reference genomes and average mapping quality values for each nucleotide were retrieved from the mapping (BAM files). Variant calling was performed using an in-house R (v3.5.1) script. Nucleotides seen ≤2 times in each position and nucleotides with mean Phred quality score of <20 were filtered out. Both F and R nucleotide counts from the same sample, obtained independently in separate amplicon reactions, were combined and variant allele frequencies were calculated for each position. If the separate reactions were discordant, the highest covered variant were used. Positions with coverage <100× were filtered out. Variants were called if variant frequency was >1%.

All nucleotide substitutions were classified into six base substitutions, C>A, C>G, C>T, T>A, T>C, and T>G, and then into 96 trinucleotide substitution types that include information on the bases immediately 5′ and 3′ of the mutated base. Analysis was performed using an in-house R (v3.5.1) script.

The statistical analyses and visualization were done in R (v3.5.1) and with the ggplot2 R package.

### Results

Characteristics and sequencing statistics of the study samples. The present study included 232 cytological cell samples from the biobank and which were categorized according to cytology/histology diagnosis of the women. A total of 80 HPV16 positive samples and 51 HPV18 positive samples passed the strict sequencing depth criteria necessary for further analyses of integration and minor nucleotide variation (Table 3). Few normal samples passed the required sequencing depth requirement and this group were therefore analyzed combined with ASC-US/LSIL. Each nucleotide in the genomes was on average sequenced 54330 times. In total 1.04 billion read pairs were analyzed. The mean sequencing coverage in the groups ranged from 4711 (CIN2) to 20850 (cancer) for HPV16 positive samples and from 147747 (CIN3/AIS) to 431649 (CIN2) for HPV18 positive samples. On average 67.2% of the genomes had a minimum of 100× coverage.

TABLE 3
Number of samples in each diagnosis group, and mean mappings
statistics in HPV16 and HPV18 positive samples.

| | | | | | Mean numbers in the analysed samples | | | |
|---|---|---|---|---|---|---|---|---|
| | | | | | | Reads | | Fraction |
| | | | | | | mapped | | of genome |
| | Mean | Sequenced | Analysed | | Raw | to target | Mean | covered |
| Diagnosis | age | samples | samples | | reads | HPV | coverage | by min. 100× |
| HPV16 | | | | | | | | |
| Normal^{a} | 49 (32-68) | 24 | 2^{e} | 21 | 1.4M | 1.1M | 13516 | 0.78 |
| ASC-US/LSIL^{a, b} | 33 (19-54) | 31 | 19^{e} | | | | | |
| CIN2^{c} | 31 (17-61) | 47 | | 27 | 0.6M | 0.4M | 4711 | 0.69 |
| CIN3/AIS^{c} | 34 (22-54) | 50 | | 27 | 1.0M | 0.8M | 9616 | 0.76 |
| Cancer^{c, d} | 30 (25-39) | 5 | | 5 | 2.4M | 1.7M | 20850 | 0.67 |
| HPV18 | | | | | | | | |
| Normal^{a} | 49 (47-52) | 3 | 1^{e} | 12 | 38.8M | 23.4M | 292143 | 0.86 |
| ASC-US/LSIL^{a, b} | 33 (20-49) | 13 | 11^{e} | | | | | |
| CIN2^{c} | 34 (20-44) | 13 | | 9 | 77.1M | 36.5M | 431649 | 0.86 |
| CIN3/AIS^{c} | 34 (24-54) | 46 | | 30 | 25.5M | 12.2M | 147747 | 0.82 |
| Cancer | — | 0 | | — | | | | |

^{a}By cytology
^{b}By cytology; no cell abnormalities within 4-years follow-up
^{c}Cytology taken at the time of the histological diagnosis
^{d}Includes cases of squamous cell carcinoma and adenocarcinoma
^{e}Samples combined for statistical analysis

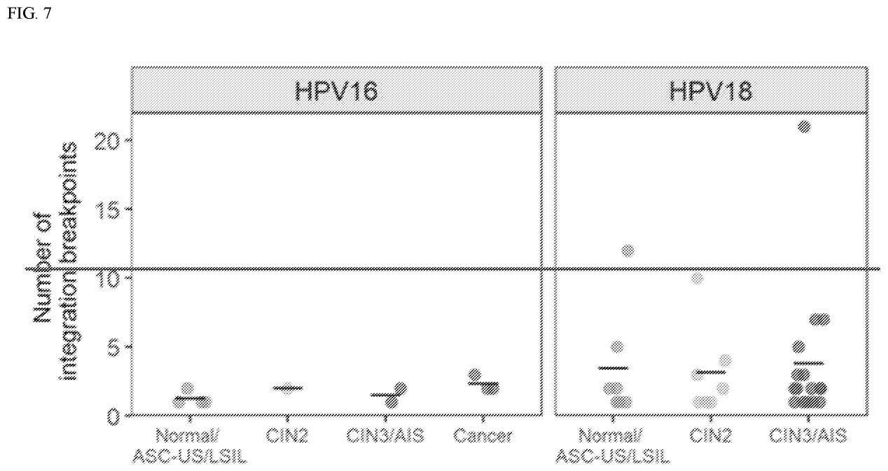

Higher HPV integration rates in HPV18 positive samples compared to HPV16 positive samples. The integration frequency was higher for all HPV18 positive morphological categories compared to the HPV16 categories (Table 4). Of the HPV16 positive samples, HPV integration was detected in 4%, 7% and 60% in CIN2, CIN3 and cancer samples, respectively. Corresponding numbers for HPV18, was 78% and 53% for CIN2 and CIN3, respectively. HPV18 positive samples also had a higher number of multiple integrations per sample. The total number of integration sites found in each morphological category was in general higher for HPV18 positive samples, ranging from 22 (CIN2) to 61 (CIN3/AIS), while for HPV16, a total of 17 integration sites were identified. The mean number of integration breakpoints per HPV18 positive sample were 3.4, 3.1 and 3.8 for normal/ASC-US/LSIL, CIN2 and CIN3/AIS groups, respectively. The mean number of integration breakpoints per HPV16 positive sample with detected integration were 1.3, 2, 1.5 and 2.3 for normal/ASC-US/LSIL, CIN2, CIN3/AIS and cancer groups, respectively (FIG. 7).

The validation rates using Sanger sequencing (good quality chromatograms produced) was 44% (Data not shown). A PCR product or a smear was identified on agarose gel but no clean chromatogram was seen in 44% of the reactions (Data not shown). Two integration sites, in HPV16 and HPV18 positive ASC-US/LSIL samples, could not be confirmed (Data not shown).

TABLE 4
Number of HPV16 and HPV18 positive samples with integration,
stratified by morphological categories

| | Number of samples with | | | | Total number |
|---|---|---|---|---|---|
| | No integration | | Integration | | of integration |
| Diagnosis | (Frequency %) | | (Frequency %) | | sites |
| HPV16 | | | | | |
| Normal/ASC-US/LSIL | 17 | (81%) | 4 | (19%) | 5 |
| (n = 21) | | | | | |
| CIN2 (n = 27) | 26 | (96%) | 1 | (4%) | 2 |
| CIN3/AIS (n = 27) | 25 | (93%) | 2 | (7%) | 3 |
| Cancer (n = 5) | 2 | (40%) | 3 | (60%) | 7 |
| HPV18 | | | | | |
| Normal/ASC-US/LSIL | 5 | (42%) | 7 | (58%) | 24 |
| (n = 12) | | | | | |
| CIN2 (n = 9) | 2 | (22%) | 7 | (78%) | 22 |
| CIN3/AIS (n = 30) | 14 | (47%) | 16 | (53%) | 61 |

| | | | |
|---|---|---|---|
| Cancer (n = 0) | — | — | — |

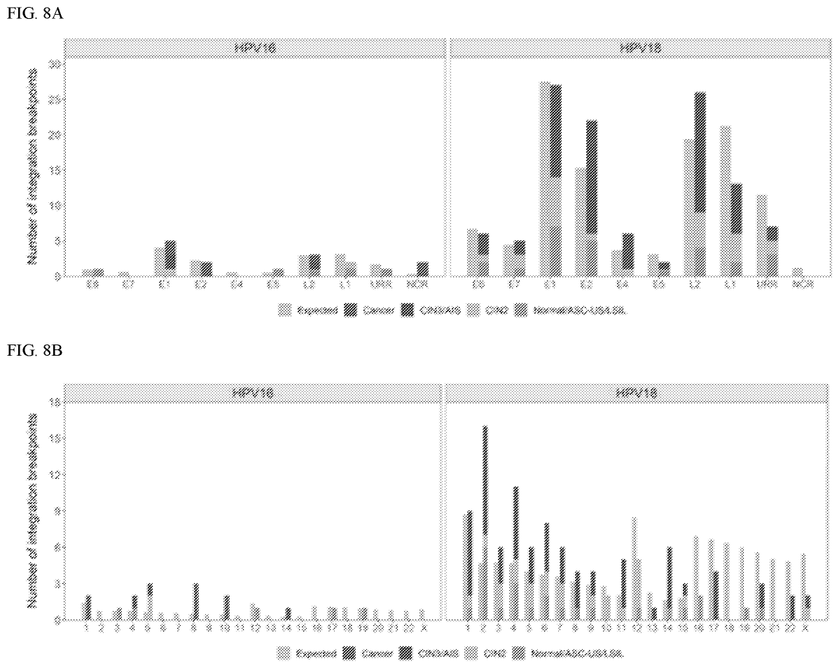

HPV breakpoints and deletions. For HPV16, breakpoints in the viral genome was detected in all genes except E4 and E7. Remarkably, the non-coding region (NCR) between the E5 and L2 genes, harbored two integration breakpoints in one cancer sample (FIG. 8a). In the HPV18 positive samples, integration breakpoints were located in all HPV genomic regions except NCR. We estimated the number of integrations that would occur in each gene, relative to gene lengths, if they occurred randomly in the genome. Integration was more frequently observed in E2, E4 and L2 than expected if the integration happened randomly. L1 and URR were less prone to integration events than expected (FIG. 8a). Integrations with breakpoints in E1, E2 or URR was observed in about 50% of the integrations in all diagnostic categories highest number of instances for an individual sample within each category was 14 in CIN3, 6 in CIN2 and 9 in ASC-US/LSIL. All the samples in the cancer group had at least one integration of this type.

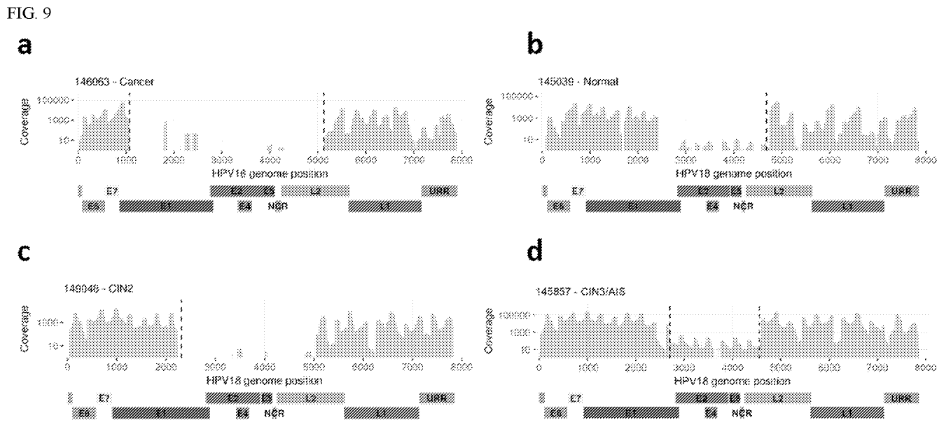

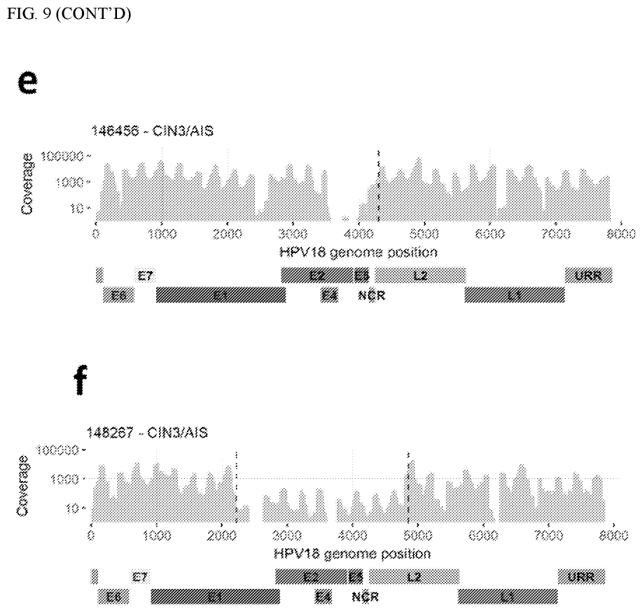

Regions covered with very few or no sequencing reads were considered as HPV genomic deletions according to previous validations (i.e., by TaMe-Seq as described herein). Such deletions were observed in six samples (Error! Reference source not found. 9). For these samples, human sequences were detected flanking the deleted regions, indicating chromosomal integration. Deletions were detected in one HPV16 positive cancer sample and in five HPV18 positive samples (FIG. 9). In all six samples, the genomic deletion encompassed the region between E1/E2 and L2. The deletions were complete (no reads detected in the deleted region) or partial, suggesting the presence of episomal HPV DNA in addition to integrated HPV DNA.

HPV integration breakpoints in the human genome. In HPV 16 positive samples, integration sites were limited to a few chromosomes; all integrations in cancer samples (n=5) were located in chromosomes 1, 8 and 10 (FIG. 8b). Interestingly, the integration breakpoints (n=3) in chromosome 8 were located in the PVT1 oncogene (Data not shown). The PVT1 gene is located in the chromosomal locus 8q24.21 (Data not shown), a HPV integration hotspot⁷⁰. In the HIPV18 positive samples, integration sites were found in all chromosomes except chromosomes 22 and X (FIG. 8b). Chromosomes 2 and 4 harbored large numbers of integration breakpoints. In chromosome 4, 31% (4/13) of integrations were located in the HPV integration hotspot chromosomal locus 4q13.3⁷⁰, and these four integrations all came from samples diagnosed with CIN2 and CIN3.

About 50% of the integrations were located in sequences of human genes, with the highest rate of 71.4% observed in cancers. The percentage of integrations in exons (CDS of genes) was highest in the normal category.

Integrations into ncRNA regions, both intergenic and genic, increased by pathology. Also the frequency of integrations occurring near cancer-related genes increased with severity of diagnosis. The lowest frequency of integrations into cancer-related genes were identified in the normal category and CIN2. In the CIN3 and cancer categories, 60% (38/63) and 100% of the integrations was detected in cancer-related genes, respectively.

We also investigated regulatory regions, including antisense, UTR and retained introns. The diagnosis group had similar proportions of integrations into these features. However, retained introns had more integrations in cancer samples compared to the other diagnosis groups.

For the samples with multiple integrations, several were identified in or in the vicinity of cancer-related genes. The maximum number of integrations observed was 21 for one CIN3 sample, of which 11 had breakpoints in or near oncogenes. In the normal category, the highest number of integrations was 11 and four of them had breakpoints near oncogenes. The highest number of integrations in the CIN2 group was 10, where 2 of them were in or near oncogenes. One interesting observation is that most of the samples with multiple integrations have integrations in or near at least one cancer-related gene.

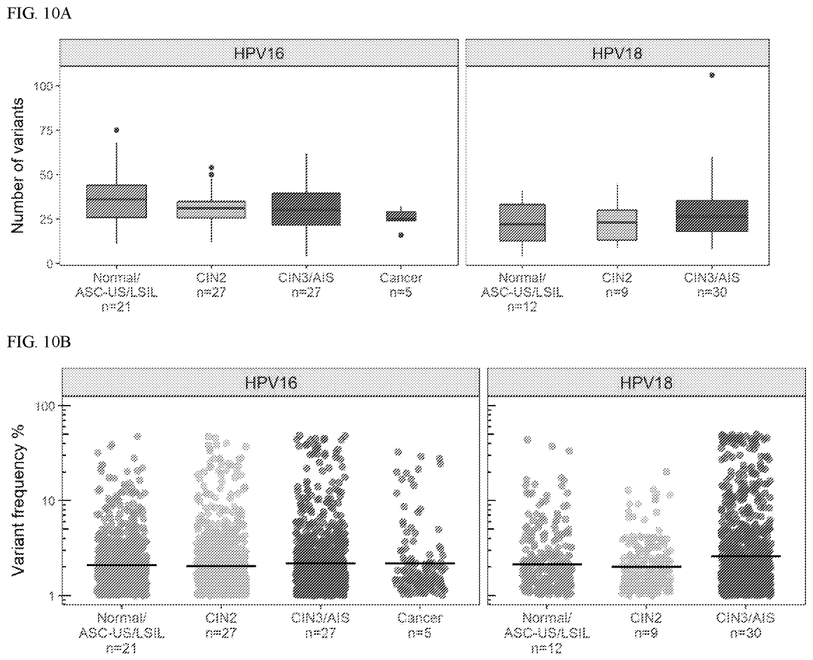

Minor nucleotide variation profiles are similar in HPV 16 and HPV 18. Overall, the number of nucleotides with variation is similar in HPV16 and HPV18 positive samples, and between the diagnostic categories (FIG. 10a). In HPV16 positive samples, 37 variants were found in the control group, 32 in the CIN2 category, 32 in the CIN3/AIS category, and 25 in the cancer category. Corresponding numbers for HPV18 positive samples were 24, 24, and 29 for normal, CIN2 and CIN3/AIS, respectively (FIG. 10a).

The mean minor nucleotide variant frequencies were slightly higher in HPV18 compared to HPV16. HPV16 positive samples had mean frequencies of 2.9% for normal, 3.1% for CIN2, 3.6% for CIN3/AIS and 3.7% for cancer samples. For HPV18 positive samples, the mean minor variant frequencies were 3.1% for normal, 2.6% for CIN2 and 5.2% for CIN3/AIS (FIG. 10b).

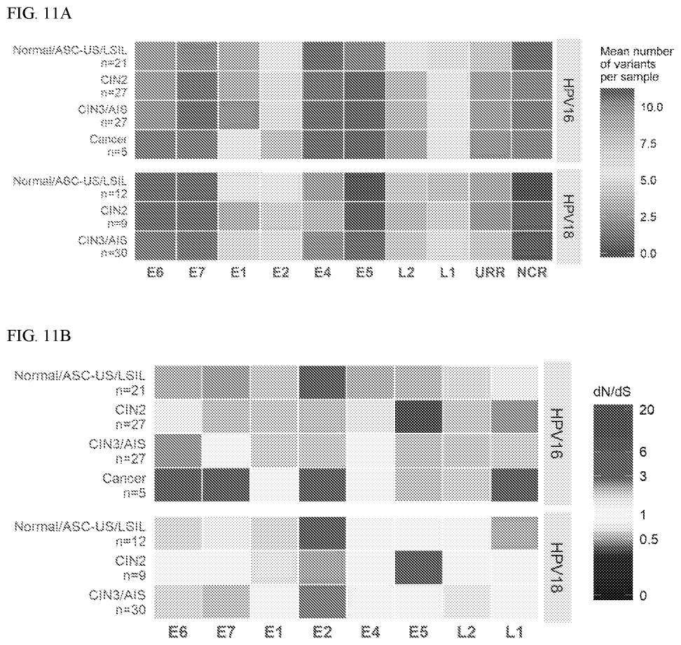

HPV variants occurred throughout all HPV genes (FIG. 11a).

Dissimilar nonsynonymous to synonymous variant ratios in HPV16 and HPV18. The ratio of non-synonymous to synonymous substitutions (dN/dS) was calculated (FIG. 11) to indicate potential selection in terms of protein-coding genes. The dN/dS patters for HPV16 showed mostly nonsynonymous variants (dN/dS >1), while a considerable part of HPV18 genes had equal amounts of nonsynonymous and synonymous variants (dN/dS≈1)) (FIG. 11b). Strikingly, the HPV16 cancer samples had a dN/dS ratio of 6 in the E6 gene, indicating positive selection on the gene (new variants favored). On the contrary, the E7 gene in the same samples had a dN/dS ratio of 0.4, indicating neutral/no selection. In HPV18 positive samples, most nonsynonymous substitutions were observed in the E2 gene, with a dN/dS ratio of >2 in all diagnosis groups; for the other genes, the dN/dS ratio was close to 1 across the diagnosis groups.

APOBEC3-related mutational signatures identified in normal and precancerous samples. Among nucleotide substitutions, C>T and T>C substitutions were predominantly observed across all diagnostic categories (FIG. 12). The APOBEC-related C>T substitutions were compared between the different categories and HPV types (Error! Reference source not found.). C>T substitutions in the trinucleotide context TCN (N is any nucleotide), a preferred target sequence for the APOBEC3 proteins⁷¹, was the most prevalent mutational signature type in HPV16 normal samples and to a slightly less extent in HPV16 CIN2 samples. HPV16 CIN3/AIS and cancer samples did not show any preferred signature patterns. Interestingly, HPV18 samples showed different C>T trinucleotide substitution patterns compared to HPV 16 samples. In all HPV18 diagnostic categories, C>T substitutions in the trinucleotide context ACA was predominantly observed, while C>T substitutions in the trinucleotide context GCA was the second most prevalent in normal/ASC-US/LSIL and CIN2 samples.

### Discussion

For a slow evolving virus, it is becoming clear that numerous within-host genomic events occur in the HPV genome. This study adds to the growing evidence of within-host HPV genome variability. When comparing Norwegian cervical samples of different morphology, we find that the genomic events are strikingly different between HPV 16 and HPV18 positive samples, but also differ dependent of lesion severity. The comprehensive, in depth analyses of 131 samples using the new TaME-seq protocol described herein, showed that integration in cancer related genes increased in HPV18 positive samples and APOBEC-related variations decreased in HPV16 positive samples with increasing lesion severity.

### REFERENCES

1 Walboomers, J. M. et al. Human papillomavirus is a necessary cause of invasive cervical cancer worldwide. J. Pathol. 189, 12-19, doi:10.1002/(sici)1096-9896(199909)189:1<12::aid-path431>3.0.co;2-f (1999).
2 Ferlay, J. et al. Cancer incidence and mortality worldwide: sources, methods and major patterns in GLOBOCAN 2012. Int. J. Cancer 136, E359-386, doi:10.1002/ijc.29210 (2015).
3 Fitzmaurice, C. et al. The Global Burden of Cancer 2013. JAMA Oncol 1, 505-527, doi:10.1001/jamaoncol.2015.0735 (2015).
4 Bosch, F. X., Lorincz, A., Munoz, N., Meijer, C. J. & Shah, K. V. The causal relation between human papillomavirus and cervical cancer. J. Clin. Pathol. 55, 244-265 (2002).
de Sanjose, S. et al. Human papillomavirus genotype attribution in invasive cervical cancer: a retrospective cross-sectional worldwide study. The Lancet Oncology 11, 1048-1056, doi:10.1016/s1470-2045(10)70230-8 (2010).
6 Crosbie, E. J., Einstein, M. H., Franceschi, S. & Kitchener, H. C. Human papillomavirus and cervical cancer. The Lancet 382, 889-899, doi:10.1016/s0140-6736(13)60022-7 (2013).
7 Forman, D. et al. Global burden of human papillomavirus and related diseases. Vaccine 30 Suppl 5, F12-23, doi:10.1016/j.vaccine.2012.07.055 (2012).
8 Moscicki, A. B. et al. Updating the natural history of human papillomavirus and anogenital cancers. Vaccine 30 Suppl 5, F24-33, doi:10.1016/j.vaccine.2012.05.089 (2012).
9 Bernard, H. U. Taxonomy and phylogeny of papillomaviruses: an overview and recent developments. Infect. Genet. Evol. 18, 357-361, doi:10.1016/j.meegid.2013.03.011 (2013).
Bzhalava, D., Eklund, C. & Dillner, J. International standardization and classification of human papillomavirus types. Virology 476, 341-344, doi:10.1016/j.virol.2014.12.028 (2015).
11 Bernard, H. U. et al. Classification of papillomaviruses (PVs) based on 189 PV types and proposal of taxonomic amendments. Virology 401, 70-79, doi:10.1016/j.virol.2010.02.002 (2010).
12 Burk, R. D., Harari, A. & Chen, Z. Human papillomavirus genome variants. Virology 445, 232-243, doi:10.1016/j.virol.2013.07.018 (2013).
13 Cornet, I. et al. HPV16 genetic variation and the development of cervical cancer worldwide. Br. J. Cancer 108, 240-244, doi:10.1038/bjc.2012.508 (2013).
14 Mirabello, L. et al. HPV16 Sublineage Associations With Histology-Specific Cancer Risk Using HPV Whole-Genome Sequences in 3200 Women. J. Natl. Cancer Inst. 108, doi:10.1093/jnci/djw100 (2016).
15 Chan, P. K. et al. Geographical distribution and oncogenic risk association of human papillomavirus type 58 E6 and E7 sequence variations. Int. J. Cancer 132, 2528-2536, doi:10.1002/ijc.27932 (2013).
16 Chen, A. A., Gheit, T., Franceschi, S., Tommasino, M. & Clifford, G. M. Human Papillomavirus 18 Genetic Variation and Cervical Cancer Risk Worldwide. J. Virol. 89, 10680-10687, doi:10.1128/jvi.01747-15 (2015).
17 de Oliveira, C. M. et al. High-level of viral genomic diversity in cervical cancers: A Brazilian study on human papillomavirus type 16. Infect. Genet. Evol. 34, 44-51, doi:10.1016/j.meegid.2015.07.002 (2015).
18 Mirabello, L. et al. HPV16 E7 Genetic Conservation Is Critical to Carcinogenesis. Cell 170, 1164-1174 e1166, doi:10.1016/j.cell.2017.08.001 (2017).
19 Hirose, Y. et al. Within-Host Variations of Human Papillomavirus Reveal APOBEC-Signature Mutagenesis in the Viral Genome. J. Virol., doi:10.1 128/jvi.00017-18 (2018).
Dube Mandishora, R. S. et al. Intra-host sequence variability in human papillomavirus. Papillomavirus Res, doi:10.1016/j.pvr.2018.04.006 (2018).
21 zur Hausen, H. Papillomaviruses and cancer: from basic studies to clinical application. Nat. Rev. Cancer 2, 342-350, doi:10.1038/nrc798 (2002).
22 Pett, M. & Coleman, N. Integration of high-risk human papillomavirus: a key event in cervical carcinogenesis? J. Pathol. 212, 356-367, doi:10.1002/path.2192 (2007).
23 McBride, A. A. & Warburton, A. The role of integration in oncogenic progression of HPV-associated cancers. PLoS Pathog. 13, e1006211, doi:10.1371/journal.ppat.1006211 (2017).
24 Jeon, S., Allen-Hoffmann, B. L. & Lambert, P. F. Integration of human papillomavirus type 16 into the human genome correlates with a selective growth advantage of cells. J. Virol. 69, 2989-2997 (1995).
25 Doorbar, J., Egawa, N., Griffin, H., Kranjec, C. & Murakami, I. Human papillomavirus molecular biology and disease association. Rev. Med. Virol. 25 Suppl 1, 2-23, doi:10.1002/rmv.1822 (2015).
26 Ziegert, C. et al. A comprehensive analysis of HPV integration loci in anogenital lesions combining transcript and genome-based amplification techniques. Oncogene 22, 3977-3984, doi:10.1038/sj.onc.1206629 (2003).
27 Peter, M. et al. Frequent genomic structural alterations at HPV insertion sites in cervical carcinoma. J Pathol. 221, 320-330, doi:10.1002/path.2713 (2010).
28 Kraus, I. et al. The Majority of Viral-Cellular Fusion Transcripts in Cervical Carcinomas Cotranscribe Cellular Sequences of Known or Predicted Genes. Cancer Res. 68, 2514-2522, doi:10.1158/0008-5472.Can-07-2776 (2008).
29 Cullen, M. et al. Deep sequencing of HPV16 genomes: A new high-throughput tool for exploring the carcinogenicity and natural history of HPV16 infection. Papillomavirus Res 1, 3-11, doi:10.1016/j.pvr.2015.05.004 (2015).
30 Xu, B. et al. Multiplex Identification of Human Papillomavirus 16 DNA Integration Sites in Cervical Carcinomas. PLoS One 8, e66693, doi:10.1371/journal.pone.0066693 (2013).
31 Liu, Y., Lu, Z., Xu, R. & Ke, Y. Comprehensive mapping of the human papillomavirus (HPV) DNA integration sites in cervical carcinomas by HPV capture technology. Oncotarget 7, 5852-5864, doi:10.18632/oncotarget.6809 (2016).
32 Hu, Z. et al. Genome-wide profiling of HPV integration in cervical cancer identifies clustered genomic hot spots and a potential microhomology-mediated integration mechanism. Nat. Genet. 47, 158-163, doi:10.1038/ng.3178 (2015).
33 Holmes, A. et al. Mechanistic signatures of HPV insertions in cervical carcinomas. npj Genomic Medicine 1, doi:10.1038/npjgenmed.2016.4 (2016).
34 Kukimoto, I. et al. Genetic variation of human papillomavirus type 16 in individual clinical specimens revealed by deep sequencing. PLoS One 8, e80583, doi:10.1371/journal.pone.0080583 (2013).
35 Geisbill, J., Osmers, U. & Durst, M. Detection and characterization of human papillomavirus type 45 DNA in the cervical carcinoma cell line MS751. J. Gen. Virol. 78 (Pt 3), 655-658, doi:10.1099/0022-1317-78-3-655 (1997).
36 Adey, A. et al. The haplotype-resolved genome and epigenome of the aneuploid HeLa cancer cell line. Nature 500, 207-211, doi:10.1038/nature12064 (2013).
37 Akagi, K. et al. Genome-wide analysis of HPV integration in human cancers reveals recurrent, focal genomic instability. Genome Res. 24, 185-199, doi:10.1101/gr.164806.113 (2014).
38 Mincheva, A., Gissmann, L. & zur Hausen, H. Chromosomal integration sites of human papillomavirus DNA in three cervical cancer cell lines mapped by in situ hybridization. Med. Microbiol. Immunol. 176, 245-256 (1987).
39 el Awady, M. K., Kaplan, J. B., O'Brien, S. J. & Burk, R. D. Molecular analysis of integrated human papillomavirus 16 sequences in the cervical cancer cell line SiHa. Virology 159, 389-398 (1987).
40 Li, T. et al. Universal Human Papillomavirus Typing Assay: Whole-Genome Sequencing following Target Enrichment. J. Clin. Microbiol. 55, 811-823, doi:10.1 128/JCM.02132-16 (2017).
41 Baker, C. C. et al. Structural and transcriptional analysis of human papillomavirus type 16 sequences in cervical carcinoma cell lines. J. Virol. 61, 962-971 (1987).
42 Yee, C., Krishnan-Hewlett, I., Baker, C. C., Schlegel, R. & Howley, P. M. Presence and expression of human papillomavirus sequences in human cervical carcinoma cell lines. Am. J. Pathol. 119, 361-366 (1985).
43 Meissner, J. D. Nucleotide sequences and further characterization of human papillomavirus DNA present in the CaSki, SiHa and HeLa cervical carcinoma cell lines. J. Gen. Virol. 80 (Pt 7), 1725-1733, doi:10.1099/0022-1317-80-7-1725 (1999).
44 Hudelist, G. et al. Physical state and expression of HPV DNA in benign and dysplastic cervical tissue: different levels of viral integration are correlated with lesion grade. Gynecol. Oncol. 92, 873-880, doi:10.1016/j.ygyno.2003.11.035 (2004).
45 Liu, Y. et al. Genome-wide profiling of the human papillomavirus DNA integration in cervical intraepithelial neoplasia and normal cervical epithelium by HPV capture technology. Sci. Rep. 6, 35427, doi:10.1038/srep35427 (2016).
46 Li, H. et al. Preferential sites for the integration and disruption of human papillomavirus 16 in cervical lesions. J. Clin. Virol. 56, 342-347, doi:10.1016/j.jcv.2012.12.014 (2013).
47 Schirmer, M., D'Amore, R., Ijaz, U. Z., Hall, N. & Quince, C. Illumina error profiles: resolving fine-scale variation in metagenomic sequencing data. BMC Bioinformatics 17, 125, doi:10.1186/s12859-016-0976-y (2016).
48 Warren, C. J. et al. APOBEC3A functions as a restriction factor of human papillomavirus. J. Virol. 89, 688-702, doi:10.1128/JVI.02383-14 (2015).
49 Kukimoto, I. et al. Hypermutation in the E2 gene of human papillomavirus type 16 in cervical intraepithelial neoplasia. J. Med. Virol. 87, 1754-1760, doi:10.1002/jmv.24215 (2015).
50 Chen, J. & Furano, A. V. Breaking bad: The mutagenic effect of DNA repair. DNA Repair (Amst) 32, 43-51, doi:10.1016/j.dnarep.2015.04.012 (2015).
51 Lamble, S. et al. Improved workflows for high throughput library preparation using the transposome-based Nextera system. BMC Biotechnol. 13, 104, doi:10.1186/1472-6750-13-104 (2013).
52 Soderlund-Strand, A., Carlson, J. & Dillner, J. Modified general primer PCR system for sensitive detection of multiple types of oncogenic human papillomavirus. J. Clin. Microbiol. 47, 541-546, doi:10.1128/JCM.02007-08 (2009).
53 Schmitt, M. et al. Bead-based multiplex genotyping of human papillomaviruses. J. Clin. Microbiol. 44, 504-512, doi:10.1 128/JCM.44.2.504-512.2006 (2006).
54 Beaudenon, S. et al. A novel type of human papillomavirus associated with genital neoplasias. Nature 321, 246-249, doi:10.1038/321246a0 (1986).
55 Van Doorslaer, K. et al. The Papillomavirus Episteme: a central resource for papillomavirus sequence data and analysis. Nucleic Acids Res. 41, D571-578, doi:10.1093/nar/gks984 (2013).
56 Sievers, F. et al. Fast, scalable generation of high-quality protein multiple sequence alignments using Clustal Omega. Mol. Syst. Biol. 7, 539, doi:10.1038/msb.2011.75 (2011).
57 Untergasser, A. et al. Primer3—new capabilities and interfaces. Nucleic Acids Res. 40, e115, doi:10.1093/nar/gks596 (2012).
58 Kozich, J. J., Westcott, S. L., Baxter, N. T., Highlander, S. K. & Schloss, P. D. Development of a dual-index sequencing strategy and curation pipeline for analyzing amplicon sequence data on the MiSeq Illumina sequencing platform. Appl. Environ. Microbiol. 79, 5112-5120, doi:10.1128/AEM.01043-13 (2013).
59 Martin, M. Cutadapt removes adapter sequences from high-throughput sequencing reads. 2011 17, doi:10.14806/ej.17.1.200 (2011).
60 Kim, D., Langmead, B. & Salzberg, S. L. HISAT: a fast spliced aligner with low memory requirements. Nat Methods 12, 357-360, doi:10.1038/nmeth.3317 (2015).
61 Li, H. et al. The Sequence Alignment/Map format and SAMtools. Bioinformatics 25, 2078-2079, doi:10.1093/bioinformatics/btp352 (2009).
62 Kielbasa, S. M., Wan, R., Sato, K., Horton, P. & Frith, M. C. Adaptive seeds tame genomic sequence comparison. Genome Res. 21, 487-493, doi:10.1101/gr.113985.110 (2011).
63 Trope, A. et al. Performance of human papillomavirus DNA and mRNA testing strategies for women with and without cervical neoplasia. J. Clin. Microbiol. 47, 2458-2464, doi:10.1128/JCM.01863-08 (2009).
64 Trope, A. et al. Cytology and human papillomavirus testing 6 to 12 months after ASCUS or LSIL cytology in organized screening to predict high-grade cervical neoplasia between screening rounds. J Clin. Microbiol. 50, 1927-1935, doi:10.1128/JCM.00265-12 (2012).
65 Lagström, S. et al. TaME-seq: An efficient sequencing approach for characterisation of HPV genomic variability and chromosomal integration. Sci. Rep. 9, 524, doi:10.1038/s41598-018-36669-6 (2019).
66 Kozich, J. J., Westcott, S. L., Baxter, N. T., Highlander, S. K. & Schloss, P. D. Development of a dual-index sequencing strategy and curation pipeline for analyzing amplicon sequence data on the MiSeq Illumina sequencing platform. Appl. Environ. Microbiol. 79, 5112-5120, doi:10.1128/AEM.01043-13 (2013).
67 Van Doorslaer, K. et al. The Papillomavirus Episteme: a central resource for papillomavirus sequence data and analysis. Nucleic Acids Res. 41, D571-578, doi:10.1093/nar/gks984 (2013).
68 Li, H. et al. The Sequence Alignment/Map format and SAMtools. Bioinformatics 25, 2078-2079, doi:10.1093/bioinformatics/btp352 (2009).
69 Kielbasa, S. M., Wan, R., Sato, K., Horton, P. & Frith, M. C. Adaptive seeds tame genomic sequence comparison. Genome Res. 21, 487-493, doi:10.1101/gr.113985.110 (2011).
70 Kraus, I. et al. The Majority of Viral-Cellular Fusion Transcripts in Cervical Carcinomas Cotranscribe Cellular Sequences of Known or Predicted Genes. Cancer Res. 68, 2514-2522, doi: 10.1158/0008-5472.Can-07-2776 (2008).
71 Warren, C. J., Westrich, J. A., Doorslaer, K. V. & Pyeon, D. Roles of APOBEC3A and APOBEC3B in Human Papillomavirus Infection and Disease Progression. Viruses 9, doi:10.3390/v9080233 (2017).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in diagnostic assays, molecular biology, sequencing, or related fields are intended to be within the scope of the following claims.

1. A method of amplifying a target nucleic acid sequence for use in a parallel sequencing method comprising:
tagmenting a target nucleic sample to provide a plurality of tagmented sequences comprising a transposon adapter sequence at the ends of the tagmented sequences;
contacting a first sample of the tagmented sequences with 1) a tag primer comprising a tag sequence portion that anneals to the transposon adapter sequence and a tag primer adapter portion, 2) a plurality of forward primers, each forward primer comprising a target sequence portion that anneals to a preselected portion of the sense strand of the target nucleic acid sequence and a forward primer sequencing portion, and 3) a sequencing primer comprising a portion that anneals to the forward sequencing portion of the forward primer and a sequencing primer adapter portion;
performing a forward amplification reaction on the first sample of the tagmented sequences to provide a first library of amplicons spanning the target nucleic acid sequence;
contacting a second sample of the tagmented sequences with 1) a tag primer comprising a tag sequence portion that anneals to the transposon sequence and a tag primer adapter portion, 2) a plurality of reverse primers, each reverse primer comprising a target sequence portion that anneals to a preselected portion of the antisense strand of the target nucleic acid sequence and a reverse primer sequencing portion, and 3) a sequencing primer comprising a portion that anneals to the reverse sequencing portion of the forward primer and a sequencing primer adapter portion; and
performing a reverse amplification reaction on the second sample of the tagmented sequences to provide a second library of amplicons spanning the target nucleic acid sequence.

2. The method of claim 1, wherein the tag primer adapter portion of the tag primer comprises a barcode sequence and a first tail portion.

3. The method of claim 2, wherein the barcode sequence is an Illumina i5 index sequence and the first tail portion is an Illumina p5 tail.

4. The method of claim 2, wherein the barcode sequence is an Illumina i7 index sequence and the first tail portion is an Illumina p7 tail.

5. The method of claim 1, wherein the sequencing primer adapter portion of the sequencing primer comprises a barcode sequence and a second tail portion.

6. The method of claim 5, wherein the barcode sequence is an Illumina i7 index sequence and the second tail portion is an Illumina p7 tail.

7. The method of claim 5, wherein the barcode sequence is an Illumina i5 index sequence and the second tail portion is an Illumina p5 tail.

8. The method of any of claims 1 to 7, wherein the tag primers used in the forward and reverse reactions are identical.

9. The method of any of claims 1 to 7, wherein the sequencing primers used in the forward and reverse reactions are identical.

10. The method of any one of claims 1 to 9, wherein tag primers comprise an Illumina i5 index sequence and an Illumina p5 tail and the sequencing primers comprise an Illumina i7 index sequence and an Illumina p7 tail.

11. The method of any one of claims 1 to 9, wherein tag primers comprise an Illumina i7 index sequence and an Illumina p7 tail and the sequencing primers comprise an Illumina i5 index sequence and an Illumina p5 tail.

12. The method of any one claims 1 to 11, wherein the plurality of forward primers comprises from about 10 to 500 forward primers.

13. The method of claim 12, wherein the plurality of forward primers are designed so that the primers anneal to the target nucleic acid sequence at intervals of from 50 to 500 bases along the entire length of the target nucleic acid sequence.

14. The method of any one claims 1 to 11, wherein the plurality of reverse primers comprises from about 10 to 500 forward primers.

15. The method of claim 14, wherein the plurality of reverse primers are designed so that the primers anneal to the target nucleic acid sequence at intervals of from 50 to 500 bases along the entire length of the target nucleic acid sequence.

16. The method of any one of claims 1 to 15, wherein the target nucleic sequence is from 1000 to 100000 bases in length.

17. The method of any of claims 1 to 16, wherein the target nucleic sequence is an integrated viral sequence.

18. The method of claim 17, wherein the integrated viral sequence is Human Papillomavirus (HPV) sequence.

19. The method of claim 17, wherein the integrated viral sequence is a Human Immunodeficiency Virus (HIV).

20. The method of any one of claims 17 to 19, wherein the tagmentation reaction produces fragments that span the 5′ and 3′ integration sites of the integrated viral sequence so that after amplification the library contains amplicons that span the 5′ and 3′ integration sites of the integrated viral sequence.

21. The method of any one of claims 1 to 20, further comprising the step of sequencing the libraries of amplicons.

22. The method of claim 21, wherein the libraries are pooled for sequencing.

23. The method of claims 21 and 22, wherein the libraries are sequenced by massively parallel sequencing.

24. A kit or system for amplifying tagmented target nucleic acid tagged with a transposon adapter sequence in preparation for sequencing comprising:
a tag primer comprising a tag sequence portion that anneals to the transposon adapter sequence and a tag primer adapter portion,
a plurality of forward primers, each forward primer comprising a target sequence portion that anneals to a preselected portion of the sense strand of the target nucleic acid sequence and a forward primer sequencing portion,
a plurality of reverse primers, each reverse primer comprising a target sequence portion that anneals to a preselected portion of the antisense strand of the target nucleic acid sequence and a reverse primer sequencing portion,
a sequencing primer comprising a portion that anneals to the forward and reverse sequencing portion of the forward and reverse primer and a sequencing primer adapter portion.

25. The kit or system of claim 24, wherein the tag primer adapter portion of the tag primer comprises a barcode sequence and a first tail portion.

26. The kit or system of claim 25, wherein the barcode sequence is an Illumina i5 index sequence and the first tail portion is an Illumina p5 tail.

27. The kit or system of claim 25, wherein the barcode sequence is an Illumina i7 index sequence and the first tail portion is an Illumina p7 tail.

28. The kit or system of claim 24, wherein the sequencing primer adapter portion of the sequencing primer comprises a barcode sequence and a second tail portion.

29. The kit or system of claim 28, wherein the barcode sequence is an Illumina i7 index sequence and the second tail portion is an Illumina p7 tail.

30. The kit or system of claim 28, wherein the barcode sequence is an Illumina i5 index sequence and the second tail portion is an Illumina p5 tail.

31. The kit or system of any of claims 24 to 30, wherein the tag primers used in the forward and reverse reactions are identical.

32. The kit or system of any of claims 24 to 30, wherein the sequencing primers used in the forward and reverse reactions are identical.

33. The kit or system of any one of claims 24 to 32, wherein tag primers comprise an Illumina i5 index sequence and an Illumina p5 tail and the sequencing primers comprise an Illumina i7 index sequence and an Illumina p7 tail.

34. The kit or system of any one of claims 24 to 32, wherein tag primers comprise an Illumina i7 index sequence and an Illumina p7 tail and the sequencing primers comprise an Illumina i5 index sequence and an Illumina p5 tail.

35. The kit or system of any one claims 24 to 34, wherein the plurality of forward primers comprises from about 10 to 500 forward primers.

36. The kit or system of claim 35, wherein the plurality of forward primers are designed so that the primers anneal to the target nucleic acid sequence at intervals of from 50 to 500 bases along the entire length of the target nucleic acid sequence.

37. The kit or system of any one claims 24 to 36, wherein the plurality of reverse primers comprises from about 10 to 500 forward primers.

38. The kit or system of claim 37, wherein the plurality of reverse primers are designed so that the primers anneal to the target nucleic acid sequence at intervals of from 50 to 500 bases along the entire length of the target nucleic acid sequence.

39. The kit or system of any one of claims 24 to 38, wherein the target nucleic sequence is from 1000 to 100000 bases in length.

40. The kit or system of any of claims 24 to 39, wherein the target nucleic sequence is an integrated viral sequence.

41. The kit or system of claim 40, wherein the integrated viral sequence is Human Papillomavirus (HPV) sequence.

42. The kit or system of claim 40, wherein the integrated viral sequence is a Human Immunodeficiency Virus (HIV).

43. The kit or system of any one of claims 24 to 42, further comprising a transposase.

44. The kit or system of any one of claims 24 to 43, further comprising a polymerase.

45. The kit or system of any one of claims 24 to 44, further comprising one or more buffers buffers for reactions using the transposase or polymerase.


SEQUENCE LISTING (3 sequences)

SEQ ID NO: 1 - DNA, 19 bp, Artificial sequence
  Synthetic
agatgtgtataagagacag

SEQ ID NO: 2 - DNA, 19 bp, Artificial sequence
  Synthetic
ctgtctcttatacacatct

SEQ ID NO: 3 - DNA, 21 bp, Artificial sequence
  Synthetic
agacgtgtgctcttccgatct